(19) United States
(12) Patent Application Publication
(10) Pub. No.: US 2024/0306488 A1
(43) Pub. Date: Sep. 12, 2024

(54) DIVALENT PLATINUM COMPLEX PHOSPHORESCENT OLED MATERIAL AND DEVICE COMPRISING THE SAME

(71) Applicant: GUANGDONG AGLAIA OPTOELECTRONIC MATERIALS CO., LTD, Foshan (CN)

(72) Inventors: Xinwei Wu, Foshan (CN); Huiyang Li, Foshan (CN); Lei Dai, Foshan (CN); Lifei Cai, Foshan (CN)

(21) Appl. No.: 18/656,809

(22) Filed: May 7, 2024

Related U.S. Application Data
(63) Continuation-in-part of application No. PCT/CN2022/123700, filed on Oct. 4, 2022.

(30) Foreign Application Priority Data
Nov. 10, 2021 (CN) 202111324682.8

(51) Int. Cl. H10K 85/30; C07D 401/14; C07F 15/00; C09K 11/06; H10K 50/12

(52) U.S. Cl. CPC H10K 85/346; C07D 401/14; C07F 15/0086; C09K 11/06; C09K 2211/1029; C09K 2211/185; H10K 50/12

(57) ABSTRACT

The present application relates to a high-efficiency divalent platinum complex. The present application further provides an organic electroluminescent device, including a cathode, an anode, and an organic layer. The organic layer includes one or more of a hole injection layer, a hole transport layer, a light-emitting layer, a hole blocking layer, an electron injection layer, or an electron transport layer. At least one layer of the organic layer includes a compound of formula (I).

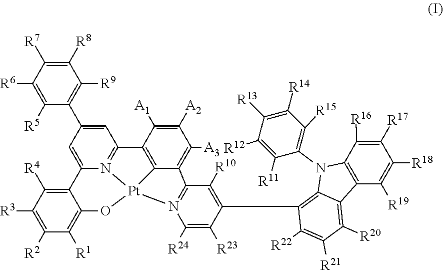

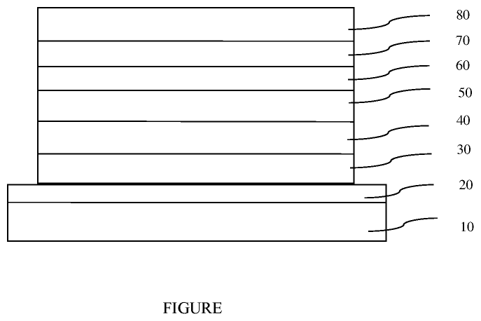

### CROSS-REFERENCE TO RELATED APPLICATIONS

This application is a continuation-in-part of PCT international patent application no. PCT/CN2022/123700 filed on Oct. 4, 2022, which itself claims priority to Chinese patent application No. 202111324682.8, filed on Nov. 10, 2021. The contents of the above identified applications are hereby incorporated herein in their entireties by reference.

### TECHNICAL FIELD

The present application relates to the field of luminescent materials, in particular to a high-efficiency tetradentate ligand-containing platinum complex and a use thereof in an organic light-emitting diode.

### BACKGROUND

With the increasing demand on informatization, new intelligent terminal products are constantly emerging. Especially, electronic products are developing gradually towards intelligent, flexible, and portable designs. The organic light-emitting diode (OLED), as a new type of display technology, have become an important focal point in the development of current material science and industrialization due to its numerous advantages such as ultralight weight and ultrathin thickness, low power consumption, self-luminous, broad operation temperature range, wide color gamut, wide viewing angle, fast response speed, and ease of realization of flexible display.

In the early fluorescent OLED, typically only the singlet state is utilized to emit lights, and the triplet excitons produced in the device cannot be effectively utilized and return to the ground state by non-radiative means, limiting the widespread use of the OLED. The phenomenon of electrophosphorescence was first reported by Zhiming Zhi, et al. at the University of Hong Kong in 1998. In the same year, a phosphorescent OLED was prepared by Thompson et al. with a transition metal complex as a luminescent material. The phosphorescent OLED can efficiently utilize singlet and triplet excitons to emit lights, with a theoretical internal quantum efficiency of 100%, greatly promoting the commercialization of the OLED. The covalent nature of metal-carbon bonds in the electrophosphorescent metal complex enhances the mixing of metal d orbitals and ligand orbitals, thereby increasing the stability of the compound. Additionally, due to the strong heavy-atom effect, the mixing of metal d orbitals and ligand orbitals can amplify the influence of the metal center on the excited states of the ligands and enhance the spin-orbit coupling effect, thereby increasing the quantum yield of triplet states and promoting efficient phosphorescent radiation relaxation. After nearly two decades of the research and development on the electrophosphorescent OLED, the focus has shifted from solely achieving efficiency breakthroughs to comprehensively enhancing both efficiency and lifespan. It is a new challenge for the research of OLED to maintain favorable properties of the multi-dimensional light-emitting device at high brightness. Addressing the issue of efficiency roll-off and shortened lifespan in the OLED device with increased brightness is crucial. The key to solve this problem lies in conducting thorough fundamental research on materials and device structures. The efficiency roll-off in the OLED device at high brightness is primarily attributed to exciton annihilation in the light-emitting layer at high current density. Especially for the phosphorescent dye, due to the long lifespan (˜μs order) of the triplet excitons thereof, triplet-triplet annihilation, triplet-singlet annihilation, and triplet-polaron annihilation are more likely to occur at the high current density, resulting in the efficiency roll-off at the high brightness. Thus, improving the luminescence kinetics of excitons is pivotal in solving this problem.

### SUMMARY

In view of the above-mentioned problems in the prior art, the present application provides a ONCN tetradentate ligand-containing platinum complex luminescent material, which exhibits a good photoelectrical property and a long device lifespan when applied to an organic light-emitting diode.

The present application further provides an organic light-emitting diode based on the platinum complex.

The ONCN tetradentate ligand-containing platinum complex is a compound having a structure of formula (I):

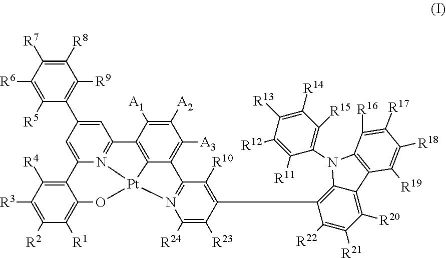

wherein:
R¹ to R²⁴ are each independently selected from hydrogen, deuterium, halogen, amino, carboxyl, thioalkyl, cyano, sulfonyl, phosphino, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, a substituted or unsubstituted alkoxy having 1 to 20 carbon atoms, a substituted or unsubstituted aryl having 6 to 30 carbon atoms, or a substituted or unsubstituted heteroaryl having 3 to 30 carbon atoms, or any two adjacent substituents are linked or fused to form a ring;
A₁ to A₃ are each independently selected from hydrogen, deuterium, halogen, amino, carboxyl, thioalkyl, cyano, sulfonyl, phosphino, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, a substituted or unsubstituted alkoxy having 1 to 20 carbon atoms, an aryl having 6 to 30 carbon atoms, or a substituted or unsubstituted heteroaryl having 3 to 30 carbon atoms, or any two adjacent substituents are linked or fused to form a ring;
a heteroatom in the heteroaryl group includes one or more of N, S, or O;
the “substituted” refers to substitution with halogen, deuterium, amino, cyano, or C1-C4 alkyl.

In an embodiment, R¹ to R²⁴ are each independently selected from hydrogen, deuterium, halogen, amino, thioalkyl, cyano, a substituted or unsubstituted alkyl having 1 to 6 carbon atoms, a substituted or unsubstituted cycloalkyl having 3 to 6 ring carbon atoms, a substituted or unsubstituted alkenyl having 2 to 6 carbon atoms, a substituted or unsubstituted alkoxy having 1 to 6 carbon atoms, a substituted or unsubstituted aryl having 6 to 12 carbon atoms, or a substituted or unsubstituted heteroaryl having 3 to 6 carbon atoms.

A₁ to A₃ are each independently selected from hydrogen, deuterium, halogen, cyano, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, an aryl having 6 to 30 carbon atoms, or a substituted or unsubstituted heteroaryl having 3 to 30 carbon atoms.

In an embodiment, R¹ to R²⁴ are each independently selected from hydrogen, deuterium, halogen, or a substituted or unsubstituted alkyl having 1 to 6 carbon atoms.

One or more of A₁ to A₃ are selected from halogen, cyano, a substituted or unsubstituted alkyl having 1 to 6 carbon atoms, a substituted or unsubstituted cycloalkyl having 3 to 12 ring carbon atoms, a substituted or unsubstituted alkenyl having 2 to 12 carbon atoms, an aryl having 6 to 30 carbon atoms, or a substituted or unsubstituted heteroaryl having 3 to 30 carbon atoms, and the rest are hydrogen.

In an embodiment, in the general formula (I), R¹ to R²⁴ are each independently selected from hydrogen, deuterium, methyl, or tert-butyl.

One or more of A₁ to A₃ are selected from methyl, fluorine, cyano, tert-butyl, phenyl, cyanophenyl, or pyridyl, and the rest are hydrogen.

In an embodiment, R¹ to R⁴ and R¹⁰ to R²⁴ of R¹ to R²⁴ are hydrogen.

In an embodiment, at least one of R⁵ to R⁹ is not hydrogen.

In an embodiment, R⁶ and R⁸ of R⁵ to R⁹ are not hydrogen, and R⁵ and R⁹ of R⁵ to R⁹ are hydrogen.

Examples of the platinum metal complex according to the present application are listed below. However, the platinum metal complex is not limited to the listed structures:

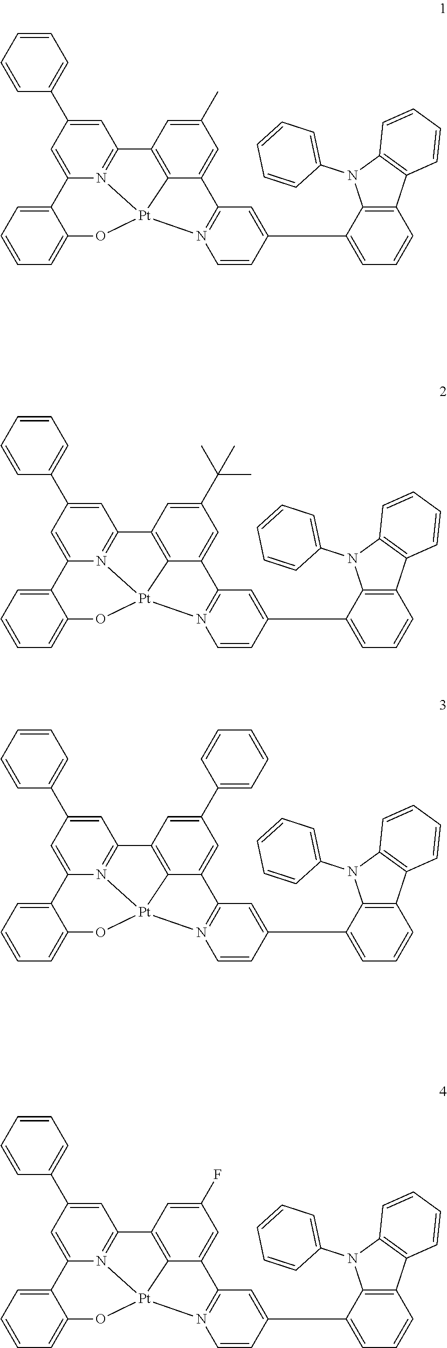

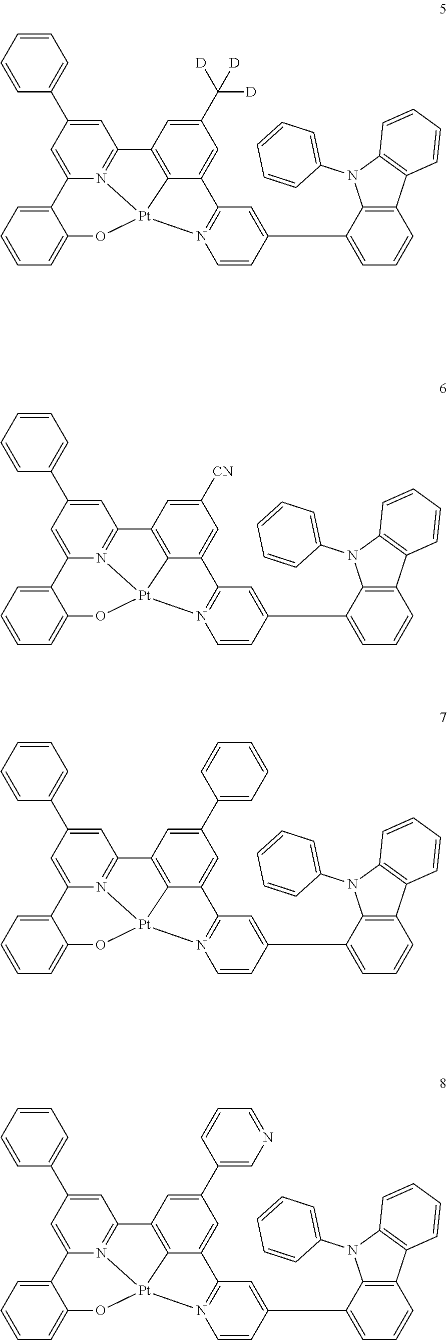

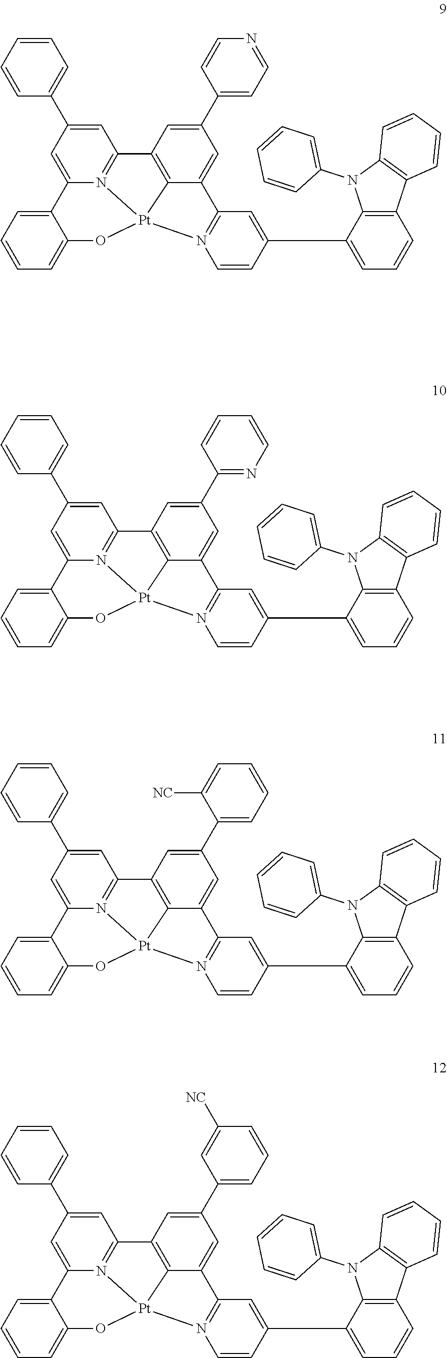

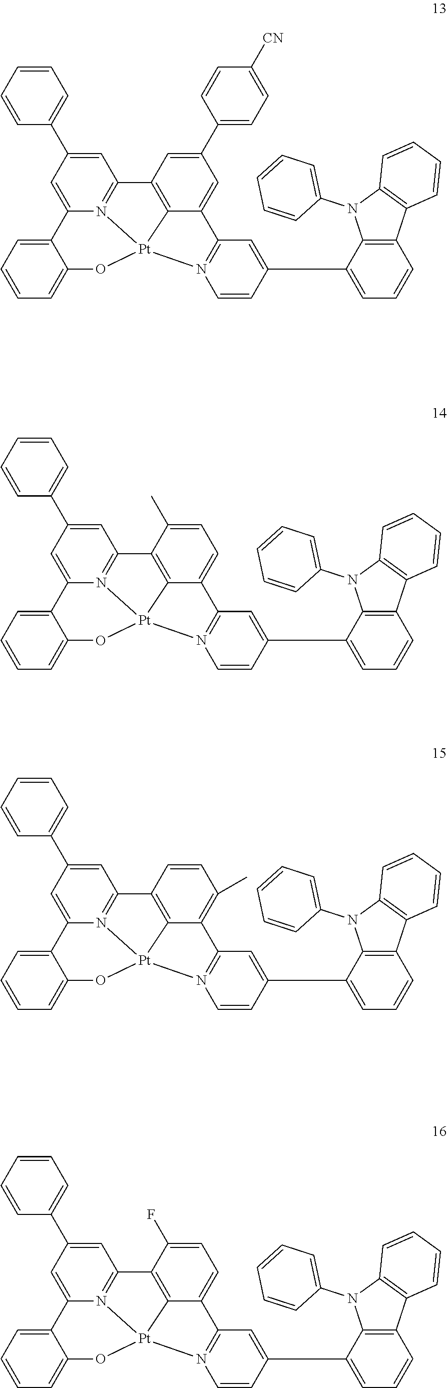

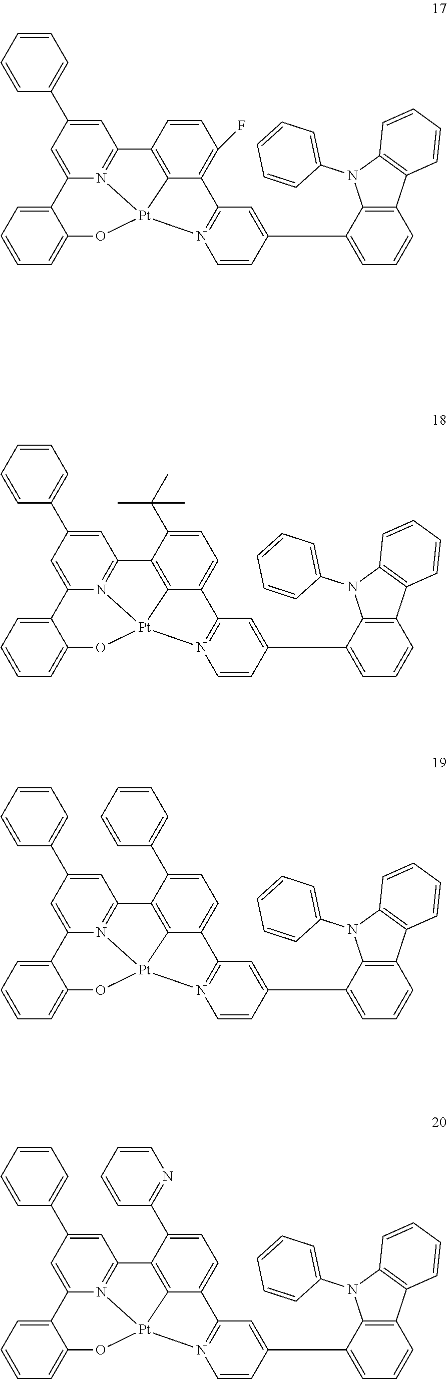

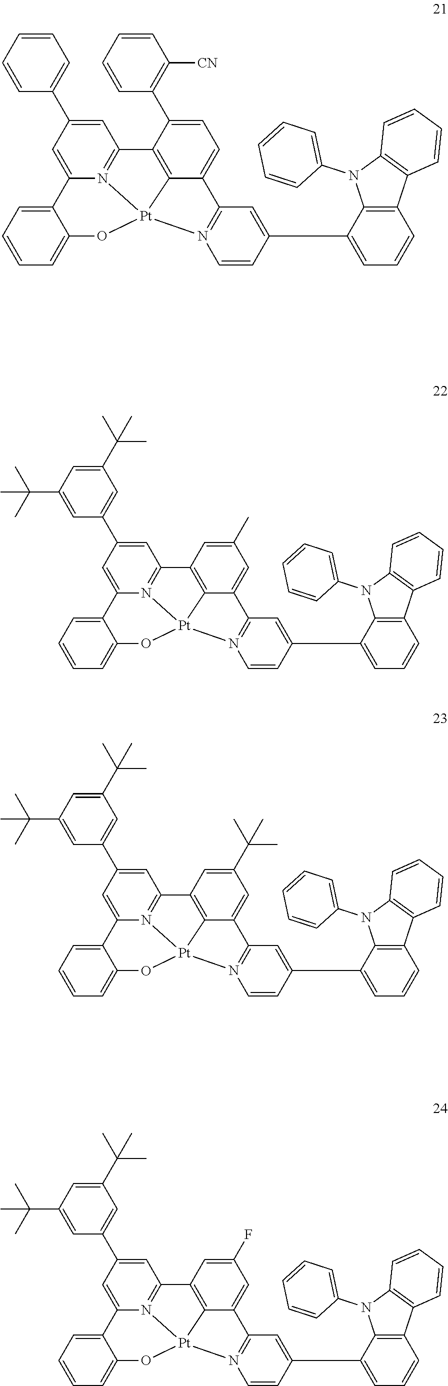

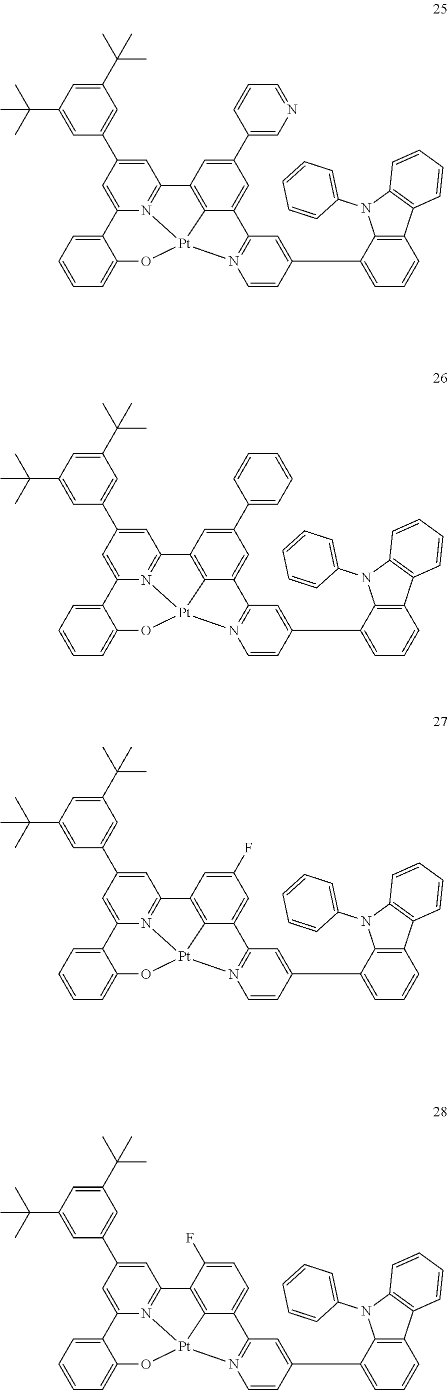

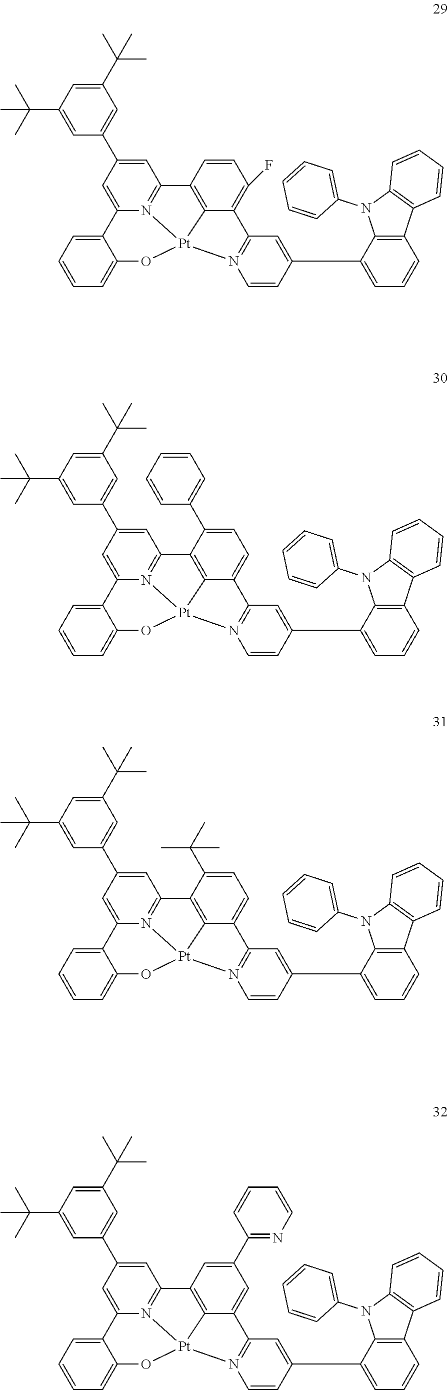

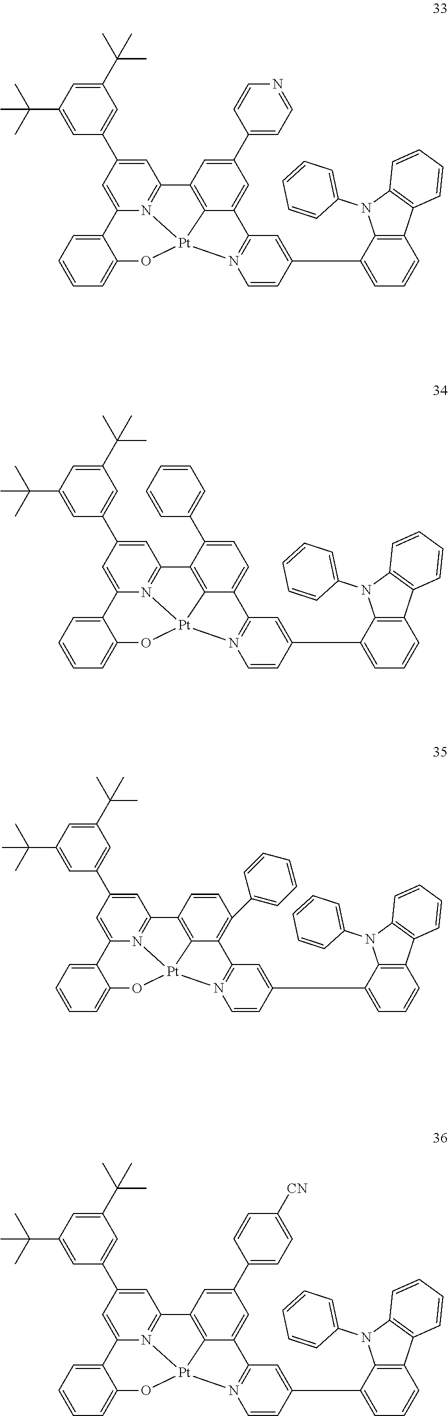

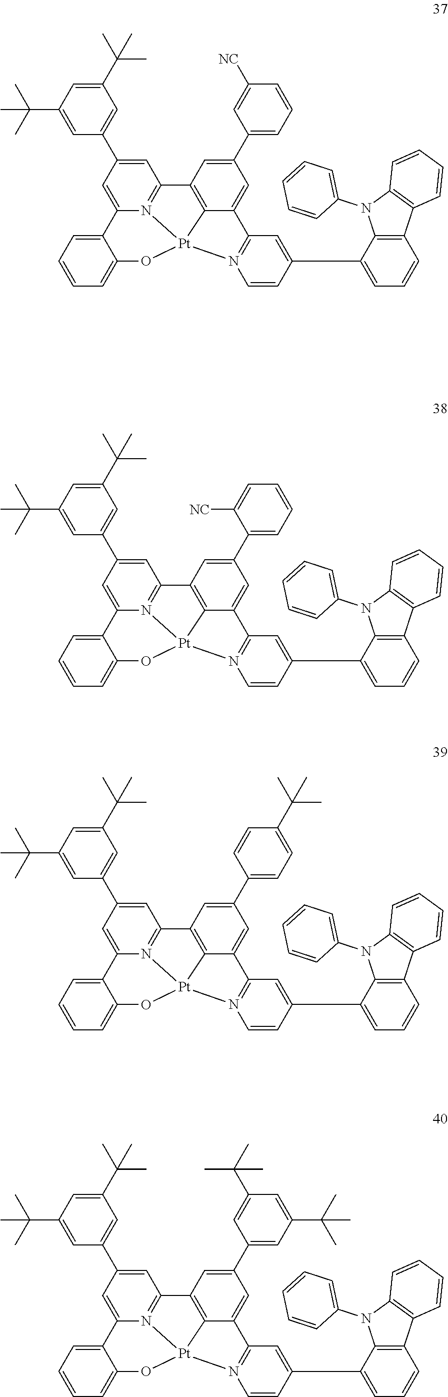

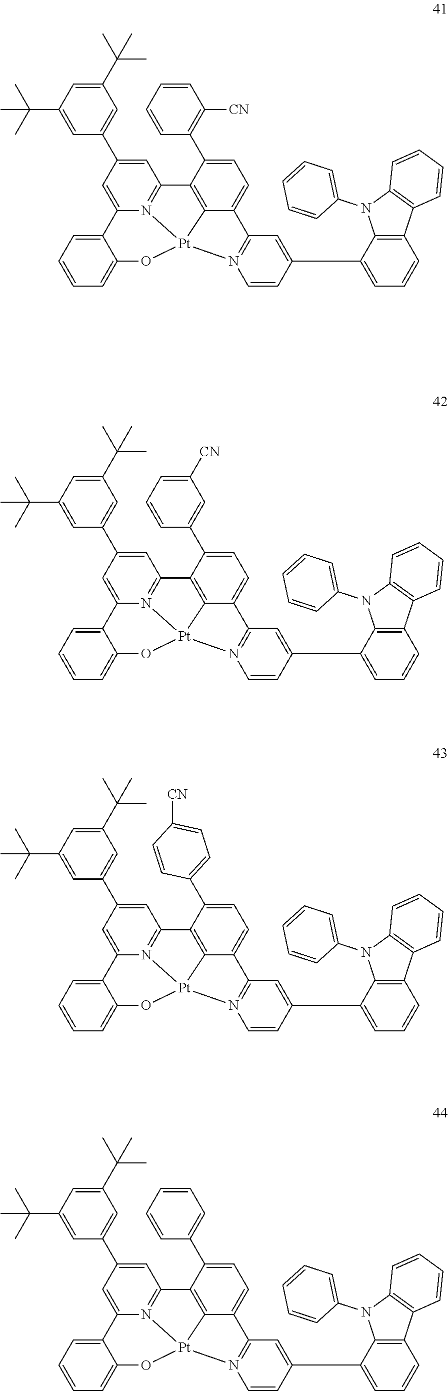

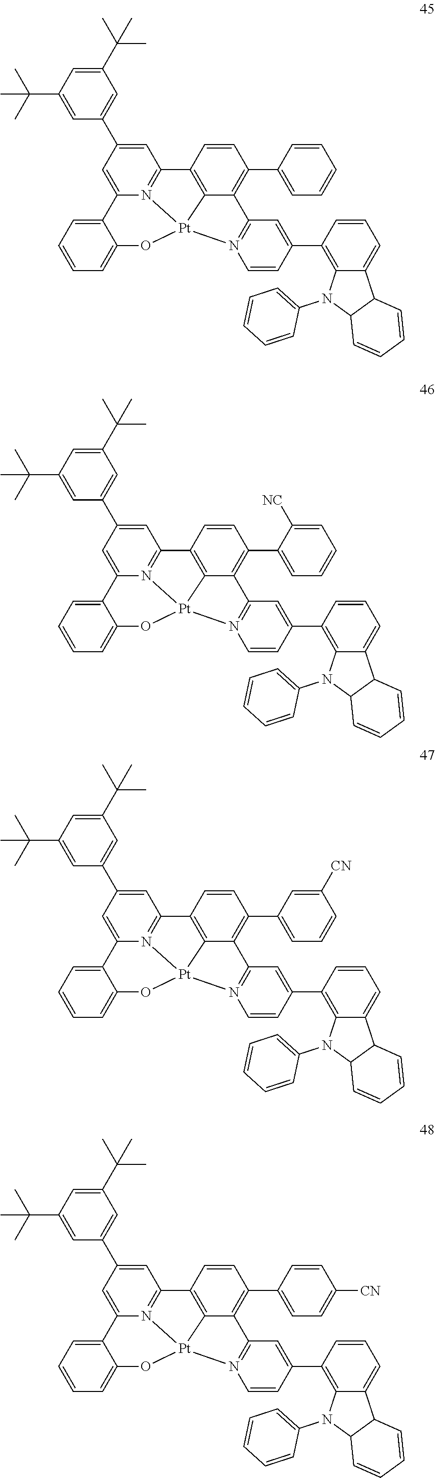

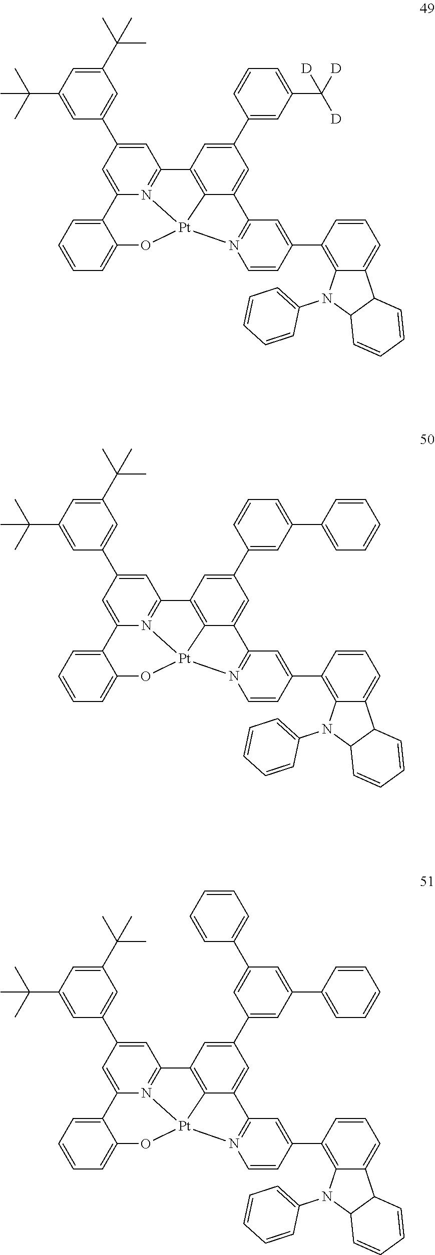

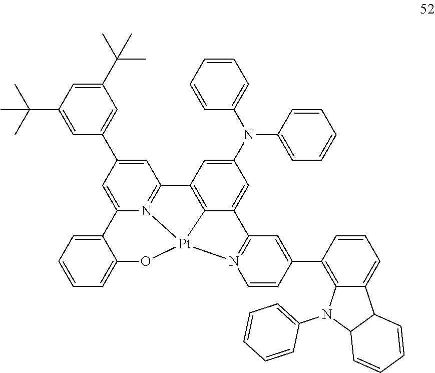

A precursor, i.e., a ligand, of the above metal complex has a structural formula as shown below:

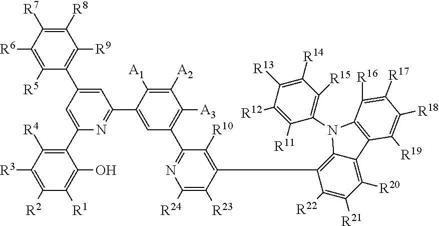

R¹ to R²⁴ and A₁ to A₃ are as defined above.

The present application further provides a use of the above platinum complex in an organic optoelectronic device. The organic optoelectronic device includes, but is not limited to, an organic light-emitting diode (OLED), an organic thin film transistor (OTFT), an organic photovoltaic (OPV), a luminescent electrochemical cell (LEC), and a chemical sensor, preferably, OLED.

An organic light-emitting diode (OLED) comprising the above platinum complex is provided. The platinum complex is used as a luminescent material of the light-emitting device.

The organic light-emitting diode of the present application includes a cathode, an anode, and an organic layer. The organic layer includes one or more of a hole injection layer, a hole transport layer, a light-emitting layer, a hole blocking layer, an electron injection layer, or an electron transport layer; and it is not necessary to provide each of these organic layers. At least one of the hole injection layer, the hole transport layer, the hole blocking layer, the electron injection layer, the light-emitting layer, or the electron transport layer comprises the platinum complex of formula (I).

In an embodiment, the layer comprising the platinum complex of formula (I) is the light-emitting layer or the electron transport layer.

The organic layer in the device of the present application has a total thickness of 1 to 1000 nm, such as, 1 to 500 nm, for example, 5 to 300 nm.

The organic layer can be a thin film formed by an evaporation method or a solution method.

The present application discloses a series of platinum complex luminescent materials with novel structures which exhibit unexpected properties. Such compound exhibits significantly improved luminous efficiency and device lifespan and good thermal stability, meeting the requirements of the OLED panel on the luminescent material.

The compound causes a low driving voltage and a high luminous efficiency and significantly increase the device lifespan when applied to an organic light-emitting diode, having a potential to be applied to the field of organic electroluminescent devices.

### BRIEF DESCRIPTION OF THE DRAWING

The FIGURE shows a structural diagram of an organic light-emitting diode device of the present application.

In the FIGURE, 10 denotes a glass substrate, 20 denotes an anode, 30 denotes a hole injection layer, 40 denotes a hole transport layer, 50 denotes a light-emitting layer, 60 denotes an electron transport layer, 70 denotes an electron injection layer, and 80 denotes a cathode.

### DETAILED DESCRIPTION

There is no requirement on the methods for synthesizing the materials in the present application. In order to describe the present application in more detail, the following examples are provided, but the present application is not limited thereto. The raw materials used in the following synthesis processes are all commercially available products unless otherwise specified.

### Example 1

### Synthesis of Complex 23

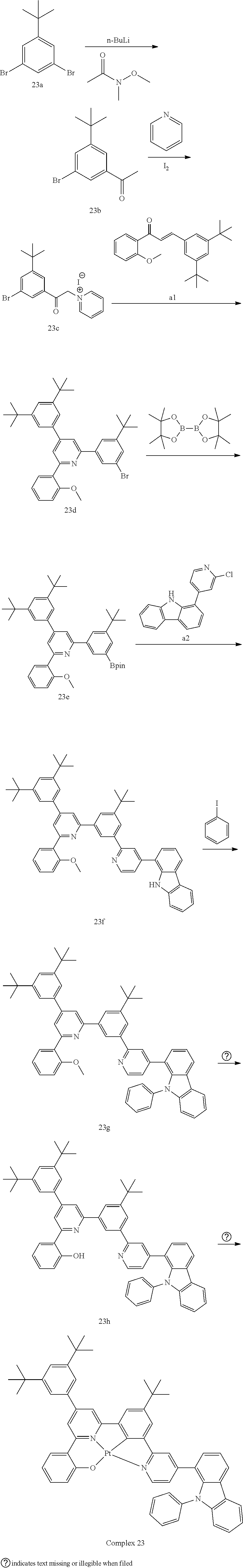

### Synthesis of Compound 23b

To a 250 ml single-neck flask, 23a (10.0 g, 34.2 mmol) and 100 mL diethyl ether were added at room temperature and N₂ was introduced. The reaction flask was placed in a low temperature reactor at −78° C. and kept for 30 min. After the temperature was stable, the reaction flask was slowly added dropwise with n-BuLi (18 mL, 37.6 mmol) through a dropping funnel and kept at −78° C. for 1 hour after the dropwise addition. Subsequently, the reaction flask was slowly added dropwise with N-methoxy-N-methylacetamide (3.85 g, 37.6 mmol) through the dropping funnel, kept for 30 minutes after the dropwise addition, slowly warmed to room temperature, and subjected to stirring overnight at room temperature. After the reaction was finished, the reaction liquid was extracted with ethyl acetate (EA)/H₂O for 3 times and subjected to a separation by silica gel column chromatography (with Hex/EA in 20/1 as an eluant) to obtain 7.0 g of a yellow oil with a yield of 77%.

¹H NMR (400 MHz, CDCl₃) δ 7.89 (d, J=10.7 Hz, 2H), 7.71 (s, 1H), 2.60 (s, 3H), 1.34 (s, 9H).

### Synthesis of Compound 23c

23b (7.0 g, 27.4 mmol), elementary iodine (7.7 g, 30.2 mmol), and pyridine (86.8 g, 1.10 mol) were added to a 250 ml single-necked flask and subjected to a reaction at 130° C. for 16 h under the protection of nitrogen. After the reaction was finished, the reaction liquid was added with 200 ml ethyl acetate to produce a yellow precipitate and suction filtered. The crude product was pulped with 250 mL ethyl acetate at 80° C. for three times, suction filtered, and oven-dried to obtain 12.5 g of a flesh-colored powder with a yield of 98%. ¹H NMR (400 MHz, DMSO) δ 8.99 (d, J=5.6 Hz, 2H), 8.72 (d, J=7.8 Hz, 1H), 8.30-8.25 (m, 2H), 8.03 (s, 1H), 7.97 (d, J=9.9 Hz, 2H), 6.55 (s, 2H), 1.32 (s, 9H).

### Synthesis of Compound 23d

23c (5.0 g, 10.96 mmol), an intermediate a1 (3.2 g, 9.13 mmol), ammonium acetate (25 g), and 50 mL acetic acid were added to a 250 ml single-necked flask. The reaction liquid was subjected to a reaction at 130° C. for 16 h under the protection of nitrogen. After the reaction was finished, the reaction liquid was extracted with EA/H₂O for several times to remove most of the acetic acid solution and the residual ammonium acetate solid, added with silica gel, subjected to a rotary evaporation, loaded into a column with pure hexane (HX or Hex), and eluted with HX/EA in 40/1 for a first pass and with HX/THF in 40/1 for a second pass. The remaining unpurified portion was further recrystallized with EA/CH₃OH for several times. 7.75 g of light yellow powder was obtained with a yield of 58%.

¹H NMR (400 MHz, CDCl₃) δ 8.07 (t, J=11.5 Hz, 4H), 7.78 (s, 1H), 7.62-7.58 (m, 2H), 7.56 (s, 2H), 7.45 (t, J=7.8 Hz, 1H), 7.19 (t, J=7.5 Hz, 1H), 7.08 (d, J=8.2 Hz, 1H), 3.94 (d, J=1.6 Hz, 3H), 1.45 (d, J=2.2 Hz, 18H), 1.41 (d, J=2.2 Hz, 9H).

### Synthesis of Compound 23e

23d (6.6 g, 10.68 mmol), B₂Pin₂ (5.81 g, 21.35 mmol), Pd(dppf)Cl₂ (0.82 g, 1.07 mmol), KOAc (3.3 g, 32.34 mmol), and DMF (120 mL) were added to a 250 ml of single-necked flask, and subjected to a reaction at 85° C. for 16 h under the protection of nitrogen. After the reaction was finished, a rotary evaporation was performed to remove most of DMF. The crude product was extracted with EA and water, dried, subjected to a rotary evaporation, and subjected to a separation by column chromatography with EA/HX in 1/10 to 1/4 as an eluent. The impure portion was further recrystallized with EA/CH₃OH. 5.0 g of a white powder was obtained with a yield of 70%.

¹H NMR (400 MHz, CDCl₃) δ 8.29 (d, J=12.5 Hz, 2H), 8.07 (dd, J=7.6, 1.6 Hz, 1H), 8.02 (d, J=1.2 Hz, 1H), 7.92 (s, 1H), 7.85 (s, 1H), 7.58-7.51 (m, 3H), 7.42 (t, J=7.0 Hz, 1H), 7.17 (t, J=7.1 Hz, 1H), 7.07 (d, J=8.3 Hz, 1H), 3.93 (s, 3H), 1.43 (d, J=5.1 Hz, 27H), 1.38 (s, 12H).

### Synthesis of Compound 23f

23e (5.0 g, 8.2 mmol), a2 (2.08 g, 7.46 mmol), Pd(PPh₃)₄ (0.382 g, 0.37 mmol), Cs₂CO₃ (7.29 g, 22.38 mmol), and toluene/ethanol/water (100/100/25 mL) were added to a 500 ml single-necked flask and subjected to a reaction at 90° C. for 48 h under the protection of nitrogen. After the reaction was finished, a rotary evaporation was performed to remove most of ethanol. The crude product was extracted with EA and water, dried, subjected to a rotary evaporation, subjected to a separation by column chromatography with EA/HX in 1/5 as an eluent, and then recrystallized with EA/CH₃OH to obtain 3.5 g of white powder with a yield of 64%.

¹H NMR (400 MHz, CDCl₃) δ 8.88 (d, J=5.1 Hz, 1H), 8.51 (d, J=11.8 Hz, 2H), 8.21 (s, 2H), 8.14 (dd, J=15.0, 7.3 Hz, 3H), 8.01 (d, J=5.3 Hz, 2H), 7.88 (s, 1H), 7.58 (d, J=4.9 Hz, 1H), 7.53 (s, 4H), 7.38 (dd, J=14.1, 6.5 Hz, 4H), 7.26 (s, 1H), 7.13-7.02 (m, 2H), 3.88 (s, 3H), 1.47 (s, 9H), 1.39 (s, 18H).

### Synthesis of Compound 23g

23f (3.5 g, 4.76 mmol), iodobenzene (2.86 g, 14.28 mmol), Cu (148.7 mg, 2.38 mmol), CuI (445.5 mg, 2.38 mmol), o-phenanthroline (843.5 mg, 4.76 mmol), Cs₂CO₃ (4.57 g, 14.28 mmol), and xylene (100 mL) were added to a 250 ml single-necked flask and subjected to a reaction at 160° C. for 24 h under the protection of nitrogen. A small amount of iodobenzene and a catalyst were supplemented and then the reaction was continued for 48 h. After the reaction was finished, the reaction liquid was extracted with EA and water, dried, subjected to a rotary evaporation, subjected to a separation by column chromatography with EA/HX in 1/4 as an eluent, and then pulped with CH₃OH at 80° C. for 3 h. 3.0 g of white powder was obtained with a yield of 79%.

¹H NMR (400 MHz, CDCl₃) δ 8.43 (d, J=4.9 Hz, 1H), 8.33 (s, 1H), 8.28 (dd, J=5.5, 3.5 Hz, 1H), 8.22 (d, J=6.6 Hz, 2H), 8.08 (dd, J=7.6, 1.6 Hz, 1H), 8.04 (d, J=1.1 Hz, 1H), 7.99 (s, 1H), 7.92 (s, 1H), 7.57 (s, 3H), 7.49-7.38 (m, 5H), 7.35 (t, J=7.3 Hz, 1H), 7.24 (d, 1H), 7.20-7.01 (m, 8H), 3.91 (s, 3H), 1.50 (s, 9H), 1.42 (s, 18H).

### Synthesis of Compound 23h

23g (2.8 g, 3.36 mmol), pyridine hydrochloride (14 g) and o-dichlorobenzene (2.8 mL) were added to a 250 ml single-necked flask and subjected to a reaction at 200° C. for 8 h under the protection of nitrogen. After the reaction was finished, the reaction liquid was extracted with EA and water, dried, subjected to a rotary evaporation, subjected to a separation by column chromatography with HX/DCM/EA=10/5/1 as an eluent, and then pulped with HX at 80° C. for 3 h. 1.85 g of a light yellow solid was obtained with a yield of 64%.

¹H NMR (400 MHz, CDCl₃) δ 8.40 (d, J=4.9 Hz, 1H), 8.29-8.24 (m, 1H), 8.20 (d, J=8.9 Hz, 2H), 8.09 (s, 1H), 8.02 (s, 1H), 7.94 (d, J=9.4 Hz, 2H), 7.87 (s, 1H), 7.59 (s, 1H), 7.51 (d, J=1.4 Hz, 2H), 7.41 (dd, J=10.4, 6.8 Hz, 4H), 7.33 (t, J=7.6 Hz, 2H), 7.27 (s, 1H), 7.07 (dd, J=16.9, 10.2 Hz, 6H), 7.02-6.92 (m, 2H), 1.49 (s, 9H), 1.41 (s, 18H).

### Synthesis of Compound 23

23h (1.7 g, 2.10 mmol), K₂PtCl₄ (1.05 g, 2.52 mmol), TBAB (135 mg, 0.42 mmol), and acetic acid (150 mL) were added to a 250 ml single-necked flask and subjected to a reaction at 130° C. for 48 h under the protection of nitrogen. During the reaction, the reaction liquid was gradually cloudy and a yellow solid was precipitated. After the reaction was finished, the reaction liquid was added with water to form a precipitate and suction filtered. The filtered solid was extracted with DCM/water, subjected to a rotary evaporation, loaded into a column, and eluted with pure DCM for a first pass to obtain a product which was subjected to a rotary evaporation and eluted with HX/DCM/EA=2/1/0.3 for a second pass. The obtained crude product was recrystallized with DCM/HX. 1.40 g of an orange-red solid was obtained with a yield of 66.67%.

¹H NMR (400 MHz, CDCl₃) δ 8.79 (d, J=5.7 Hz, 1H), 8.31 (d, J=6.5 Hz, 1H), 8.23 (d, J=7.9 Hz, 2H), 8.09 (d, J=8.2 Hz, 1H), 7.74 (s, 1H), 7.63 (d, J=9.1 Hz, 2H), 7.57 (s, 2H), 7.52-7.29 (m, 9H), 7.28 (s, 1H), 7.19 (s, 2H), 7.07 (t, J=7.8 Hz, 2H), 6.92 (s, 1H), 6.76 (s, 1H), 1.43 (s, 17H), 1.42 (s, 9H).

¹³C NMR (101 MHz, CDCl₃) δ 169.02, 168.78, 166.57, 153.46, 152.55, 152.20, 151.64, 150.91, 149.78, 148.65, 143.53, 141.69, 141.26, 141.19, 141.12, 140.91, 138.13, 137.24, 135.15, 132.13, 131.53, 131.08, 130.44, 129.81, 128.73, 127.68, 127.61, 127.16, 127.09, 126.57, 125.81, 125.54, 125.26, 124.92, 124.45, 123.98, 123.63, 123.40, 122.75, 122.56, 122.19, 121.53, 121.48, 121.21, 120.85, 120.00, 119.74, 118.84, 118.57, 118.21, 117.21, 116.54, 115.08, 114.64, 113.53, 110.54, 109.01, 108.94, 35.39, 33.35, 32.49, 32.05, 31.19, 30.76, 29.91.

ESI-MS (m/z): 1003.3 (M+1)

Those skilled in the art would be appreciated that the above preparation method is merely an illustrative example and have the ability to make modifications on the same to obtain other compound structures of the present application.

### Example 2

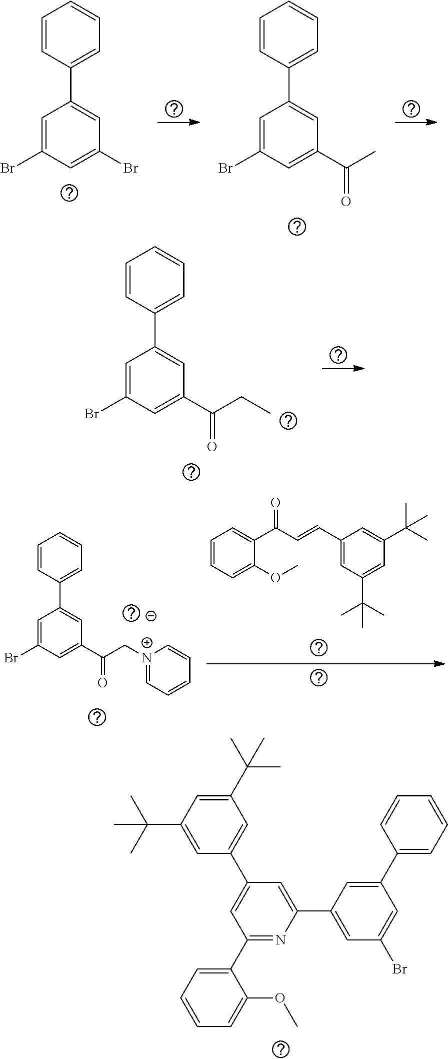 [SMILES: Brc1cc(Br)cc(-c2ccccc2)c1 ; CC(=O)c1cc(Br)cc(-c2ccccc2)c1 ; CCC(=O)c1cc(Br)cc(-c2ccccc2)c1 ; O=C(C[n+]1ccccc1)c1cc(Br)cc(-c2ccccc2)c1 ; COc1ccccc1C(=O)/C=C/c1cc(C(C)(C)C)cc(C(C)(C)C)c1 ; COc1ccccc1-c1cc(-c2cc(C(C)(C)C)cc(C(C)(C)C)c2)cc(-c2cc(Br)cc(-c3ccccc3)c2)n1]

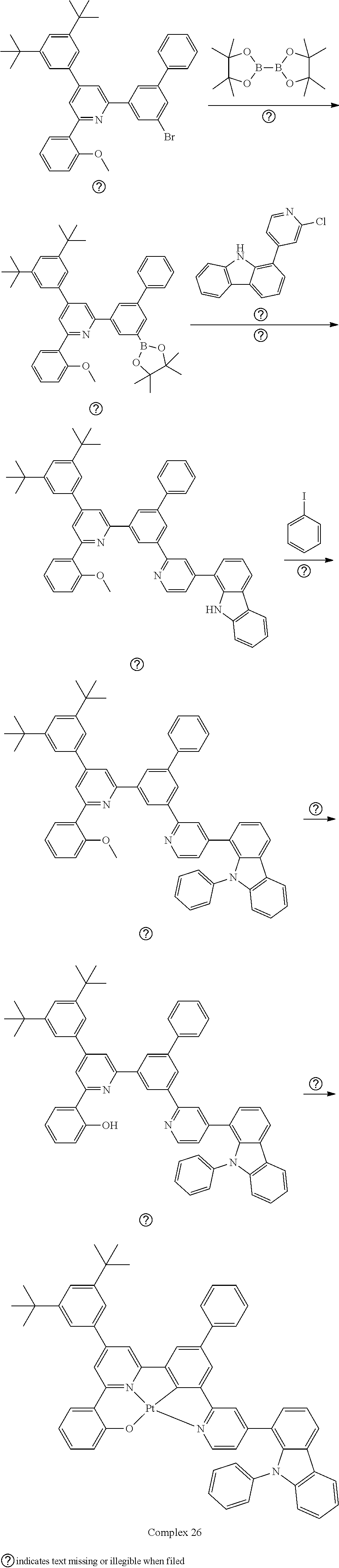

### Synthesis of Compound 26b

26a (10 g, 32.05 mmol) was dissolved in diethyl ether under the protection of nitrogen and reacted under stirring at −78° C. for 0.5 h. Then n-BuLi (24.34 ml, 1.58 M, 38.46 mmol) was slowly added dropwise followed by stirring at −78° C. for 0.5 h. Then N-methoxy-N-methylacetamide (3.97 g, 38.46 mmol) and diethyl ether (200 ml) were slowly added dropwise followed by stirring at −78° C. for 0.5 h. Then the reaction liquid was warmed to room temperature and stirred at room temperature.

After the reaction was finished, the reaction liquid was combined for treating. The reaction liquid was added with a large amount of water until a precipitate was produced, extracted with EA (100 ml) for three times, subjected to a rotary evaporation, and subjected to a separation by silica gel column chromatography (with Hex:EA=30:1 as an eluent). 18.4 g of a white solid was obtained with a yield of 69.5%.

¹H NMR (400 MHz, CDCl₃) δ 8.07 (d, J=1.5 Hz, 1H), 8.03 (t, J=1.5 Hz, 1H), 7.91 (t, J=1.6 Hz, 1H), 7.60-7.55 (m, 2H), 7.47 (t, J=7.3 Hz, 2H), 7.41 (dd, J=8.4, 6.1 Hz, 1H), 2.63 (s, 3H).

### Synthesis of Compound 26c

To a 1000 ml single-necked flask, 26b (18 g, 65.42 mmol), p-toluenesulfonic acid monohydrate (24.89 g, 130.84 mmol), and acetonitrile (400 ml) were added, NBS (11.64 g, 65.42 mmol) was added in batches in the dark, and a reaction was performed at 60° C. for 2 h under the protection of nitrogen. After the reaction was finished, the reaction liquid was rapidly filtered through a silicone funnel (with EA as an agent for drip washing), subjected to a rotary evaporation, and subjected to a separation by silica gel column chromatography (with Hex:EA=30:1 (V/V) an eluant). 18 g of a yellow oil was obtained with a field of 77.7%.

¹H NMR (400 MHz, CDCl₃) δ 8.08 (d, J=14.7 Hz, 2H), 7.95 (s, 1H), 7.58 (s, 1H), 7.56 (s, 1H), 7.48 (t, J=7.4 Hz, 2H), 7.42 (t, J=7.2 Hz, 1H), 4.50-4.42 (m, 2H).

### Synthesis of Compound 26d

26c (18 g, 50.84 mmol, 1.0 eq) and pyridine (360 ml) were added to a 1000 ml single-necked flask and subjected to a reaction at 130° C. for 6 h under the protection of nitrogen. After the reaction was finished, the reaction liquid was suction filtered. The solid was washed with EA twice and dried. 15 g of a beige solid was obtained with a yield of 68.11%.

¹H NMR (400 MHz, DMSO) δ 9.02 (d, J=5.7 Hz, 2H), 8.74 (t, J=7.8 Hz, 1H), 8.29 (t, J=7.5 Hz, 4H), 8.16 (s, 1H), 7.81 (d, J=7.3 Hz, 2H), 7.53 (t, J=7.4 Hz, 2H), 7.46 (t, J=7.3 Hz, 1H), 6.63 (s, 2H).

### Synthesis of Compound 26e

26 d (10.0 g, 28.53 mmol), a1 (13.59 g, 31.38 mmol), NH₄OAc (100 g, a ratio thereof to acetic acid=1:2), and acetic acid (200 ml) were added to a 500 ml single-necked flask and subjected to a reaction at 130° C. for 4 h under the protection of nitrogen. After the reaction was finished, the reaction liquid was added with water and extracted with DCM (50 ml) for three times. The organic phase was subjected to a rotary evaporation and a separation by silica gel column chromatography (with Hex:EA=20:1 an eluent) and then pulped with hot methanol (80° C.). 15 g of a white solid was obtained with a yield rate of 86.9%.

¹H NMR (400 MHz, CDCl₃) δ 8.26 (d, J=8.5 Hz, 2H), 8.08 (d, J=1.1 Hz, 1H), 8.04 (dd, J=7.6, 1.6 Hz, 1H), 7.85-7.78 (m, 2H), 7.66 (d, J=7.2 Hz, 2H), 7.55 (t, J=6.5 Hz, 3H), 7.48 (t, J=7.5 Hz, 2H), 7.41 (dd, J=13.4, 7.2 Hz, 2H), 7.17 (t, J=7.3 Hz, 1H), 7.07 (d, J=8.2 Hz, 1H), 3.92 (s, 3H), 1.42 (s, 18H).

### Synthesis of Compound 26f

26e (4.5 g, 7.45 mmol), bis(pinacolato)diboron (3.79 g, 14.91 mmol), Pd(OAc)₂ (16.74 mg, 0.074 mmol), x-phos (356.89 mg, 0.74 mmol), KOAc (2.19 g, 22.36 mmol), and toluene (90 mL) were added to a 250 ml three-necked flask and subjected to a reaction at 90° C. for 16 h under the protection of nitrogen.

After the reaction was finished, the reaction liquid was combined for treating. The reaction liquid was rapidly filtered through a silicone funnel (with EA as an eluant), subjected to a rotary evaporation, subjected to a separation by silica gel column chromatography (with EA as an eluant), and then pulped with hot Hex (80° C.). 8.5 g of a white solid was obtained with a yield of 87.6%.

¹H NMR (400 MHz, CDCl₃) δ 8.51 (s, 1H), 8.46 (s, 1H), 8.12 (s, 1H), 8.09-8.01 (m, 2H), 7.91 (d, J=1.2 Hz, 1H), 7.75 (d, J=7.3 Hz, 2H), 7.57-7.53 (m, 3H), 7.47 (t, J=7.6 Hz, 2H), 7.43-7.34 (m, 2H), 7.16 (t, J=7.4 Hz, 1H), 7.06 (d, J=8.2 Hz, 1H), 3.92 (s, 3H), 1.43 (s, 18H), 1.39 (s, 12H).

### Synthesis of Compound 26g

26f (2 g, 7.18 mmol, 1.0 eq), a2 (5.61 g, 8.61 mmol, 1.2 eq), Pd₂ (dba)₃ (328.53 mg, 0.36 mmol, 0.05 eq), x-phos (342.06 mg, 0.72 mmol, 0.1 eq), K₃PO₄ 3H₂O (5.73 g, 21.53 mmol, 3.0 eq), and toluene/ethanol/water (40 ml/10 ml/10 ml) were added to a 250 ml single-necked flask and subjected to a reaction at 80° C. for 4 h under the protection of nitrogen. After the reaction was finished, the reaction liquid was subjected to a rotary evaporation to remove the solvent and extracted with DCM (50 ml) for three times. The organic phase was subjected to a separation by silica gel column chromatography (with Hex:EA=8:1 an eluent) and pulped with hot Hex (80° C.). 5.2 g of a white solid was obtained with a yield rate of 94.3%.

¹H NMR (400 MHz, CDCl₃) δ 8.87 (d, J=5.0 Hz, 1H), 8.76 (d, J=14.8 Hz, 2H), 8.39 (d, J=5.9 Hz, 2H), 8.22 (s, 1H), 8.13 (dd, J=14.4, 7.7 Hz, 2H), 8.06-7.99 (m, 2H), 7.95 (s, 1H), 7.73 (d, J=7.5 Hz, 2H), 7.60-7.52 (m, 5H), 7.45 (t, J=7.6 Hz, 2H), 7.41-7.34 (m, 5H), 7.27 (dd, J=5.0, 3.1 Hz, 1H), 7.06 (dd, J=15.4, 7.9 Hz, 2H), 3.89 (s, 3H), 1.41 (s, 18H).

### Synthesis of Compound 26h

26g (5.0 g, 6.51 mmol, 1.0 eq), iodobenzene (3.98 g, 19.53 mmol, 3.0 eq), Cu (206.86 mg, 3.26 mmol, 0.5 eq), CuI (619.96 mg, 3.26 mmol, 0.5 eq), o-phenanthroline (1.17 g, 6.51 mmol, 1.0 eq), Cs₂CO₃ (6.36 g, 19.53 mmol, 3.0 eq), and xylene (100 ml) were added to a 250 ml single-necked flask and subjected to a reaction at 140° C. for 48 h under the protection of nitrogen. Iodobenzene (3.98 g, 19.53 mmol, 3.0 eq) was supplemented and the reaction was continued at 140° C. for 48 h. Iodobenzene (3.98 g, 19.53 mmol, 3.0 eq) was further supplemented and the reaction was further continued at 140° C. for 24 h. After the reaction was finished, the reaction liquid was filtered rapidly through a silicone funnel (EA), subjected to a rotary evaporation to remove the solvent, subjected to a separation by silica gel column chromatography (with Hex:DCM:EA=4:1:0.3 as an eluent), and then pulped with hot Hex (80° C.). 5 g of a white solid was obtained with a yield rate of 90.9%.

¹H NMR (400 MHz, CDCl₃) δ 8.53 (s, 1H), 8.45-8.38 (m, 2H), 8.26 (t, J=4.5 Hz, 1H), 8.20 (d, J=7.9 Hz, 2H), 8.09-8.03 (m, 2H), 7.96 (s, 1H), 7.78 (d, J=7.1 Hz, 2H), 7.55 (s, 3H), 7.50 (dd, J=14.8, 7.4 Hz, 3H), 7.40 (dd, J=13.9, 5.8 Hz, 5H), 7.33 (t, J=7.0 Hz, 1H), 7.23 (d, J=8.1 Hz, 1H), 7.15-7.01 (m, 8H), 3.89 (s, 3H), 1.40 (s, 18H).

### Synthesis of Compound 26i

26h (5 g, 5.92 mmol, 1.0 eq), pyridine hydrochloride (50 g), and o-dichlorobenzene (5 ml) were added to a 250 ml single-necked flask and subjected to a reaction at 200° C. for 5 h under the protection of nitrogen. After the reaction was finished, the reaction liquid was added with a large amount of water and extracted with DCM (30 ml) for three times. The organic phase was subjected to a separation by silica gel column chromatography (with Hex:DCM:EA=4:1:0.3 as an eluent) and then pulped with hot Hex (80° C.). 4.5 g of a yellow solid was obtained with a yield of 91.5%.

¹H NMR (400 MHz, CDCl₃) δ 8.41 (d, J=5.4 Hz, 2H), 8.30-8.25 (m, 2H), 8.24-8.18 (m, 2H), 8.06 (s, 1H), 7.99-7.92 (m, 2H), 7.79 (d, J=7.2 Hz, 2H), 7.61 (s, 1H), 7.53 (dd, J=13.4, 4.7 Hz, 5H), 7.46-7.38 (m, 4H), 7.34 (t, J=7.0 Hz, 2H), 7.28 (d, J=8.1 Hz, 1H), 7.18-7.05 (m, 6H), 7.02-6.95 (m, 2H), 1.43 (s, 18H).

### Synthesis of Complex 26

26i (1.5 g, 1.81 mmol, 1.0 eq), K₂PtCl₄ (900.13 mg, 2.17 mmol, 1.2 eq), TBAB (29.13 mg, 0.09 mmol, 0.05 eq), and acetic acid (150 ml) were subjected to a reaction at 130° C. for 48 h under the protection of nitrogen. After the reaction was finished, the reaction liquid was combined for treating. The reaction liquid was added with water to precipitate a solid and suction filtered. The solid was washed with water twice, dissolved with DCM, subjected to a separation by silica gel column chromatography with DCM as an eluent, and then subjected to a separation by silica gel column chromatography with Hex:DCM:EA=2:1:0.3 an eluent to obtain 4.2 g of red solid. The crude product was recrystallized with DCM:Hex=12 ml:12 ml to obtain 4 g of yellow solid, which was then recrystallized with DCM:MeOH=12 ml:9 ml to obtain 3.7 g of yellow solid with a yield of 66.7%.

¹H NMR (400 MHz, CDCl₃) δ 8.77 (d, J=5.8 Hz, 1H), 8.33-8.20 (m, 3H), 8.07 (d, J=8.3 Hz, 1H), 7.74 (s, 2H), 7.62 (s, 1H), 7.57 (dd, J=10.0, 4.6 Hz, 4H), 7.46-7.40 (m, 8H), 7.37 (d, J=7.9 Hz, 2H), 7.33-7.30 (m, 2H), 7.15 (d, J=4.0 Hz, 3H), 7.07 (t, J=7.9 Hz, 2H), 6.93 (d, J=7.4 Hz, 1H), 6.77 (d, J=6.1 Hz, 1H), 1.42 (s, 18H).

¹³C NMR (125 MHz, Common NMR Solvents) δ 166.22, 152.06, 151.60, 146.44, 144.39, 143.26, 140.34, 140.20, 140.07, 140.07, 139.59, 139.54, 139.32, 138.96, 138.92, 137.72, 137.14, 131.64, 130.52, 130.17, 130.07, 129.41, 129.16, 128.64, 128.33, 128.06, 127.33, 126.91, 126.87, 125.92, 125.15, 124.75, 123.93, 123.60, 123.50, 123.47, 123.44, 122.86, 122.63, 122.30, 121.69, 121.59, 120.81, 120.47, 113.76, 112.54, 34.96, 31.29.

ESI-MS (m/z): 1023.3 (M+1)

### Example 3

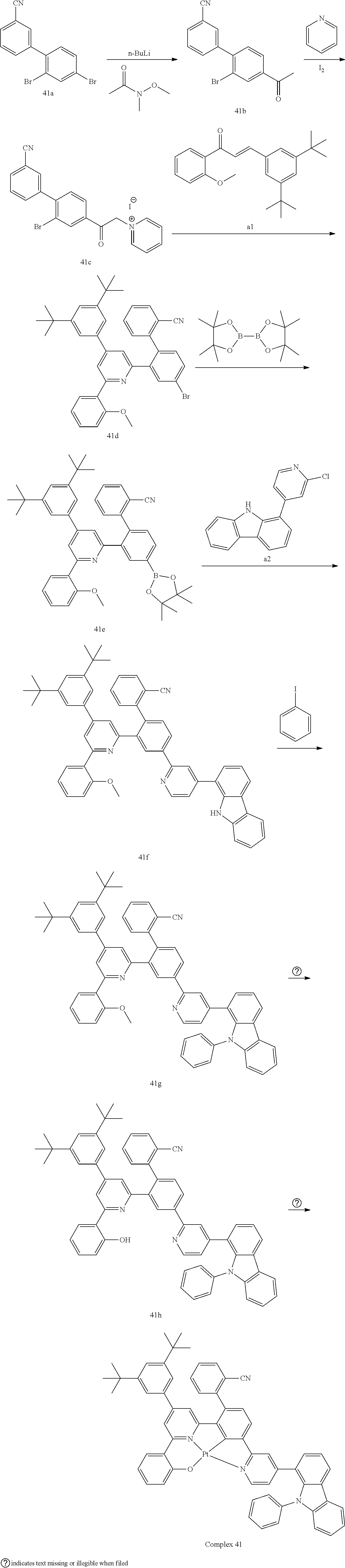

### Synthesis of Compound 41b

To a 250 ml single-neck flask, 41a (10.0 g, 29.6 mmol) and 100 mL diethyl ether were added at room temperature and N₂ was introduced. The reaction flask was placed in a low temperature reactor at −78° C. and kept for 30 min. After the temperature was stable, the reaction flask was slowly added dropwise with n-BuLi (20.8 mL, 32.6 mmol) through a dropping funnel and then kept at −78° C. for 1 hour after the dropwise addition. Subsequently, the reaction flask was slowly added dropwise with N-methoxy-N-methylacetamide (3.36 g, 32.6 mmol) through the dropping funnel, kept for 30 min after the dropwise addition, slowly warmed to room temperature, and subjected to stirring overnight at room temperature. After the reaction was finished, the reaction liquid was extracted with EA/H₂O for three times and subjected to a separation by silica gel column chromatography (with Hex/EA 20/1 as an eluent). 6.4 g of a yellow solid was obtained with a yield of 72.3%.

¹H NMR (400 MHz, CDCl₃) δ 8.38 (d, J=1.9 Hz, 1H), 7.98 (s, 1H), 7.83 (dd, J=8.3, 1.9 Hz, 1H), 7.69 (d, J=8.3 Hz, 1H), 7.66 (dt, J=6.6, 1.7 Hz, 1H), 7.62 (dt, J=6.4, 1.4 Hz, 1H), 7.58 (t, J=6.5 Hz, 1H), 2.60 (s, 3H).

### Synthesis of Compound 41c

41b (6.0 g, 20.0 mmol), elementary iodine (5.6 g, 22.0 mmol), and pyridine (63.1 g, 0.80 mol) were added to a 250 ml single-necked flask and subjected to a reaction at 130° C. for 16 h under the protection of nitrogen. After the reaction was finished, the reaction liquid was added with 200 ml ethyl acetate to produce a yellow precipitate and suction filtered. The crude product was pulped with 250 mL ethyl acetate at 80° C. for three times, suction filtered, and oven-dried. 9.8 g of a red solid was obtained with a yield of 97%. ¹H NMR (400 MHz, DMSO) δ9.13 (dt, J=5.4, 1.3 Hz, 2H), 8.65 (tt, J=7.9, 1.3 Hz, 1H), 8.27-8.20 (m, 2H), 8.04-7.97 (m, 3H), 7.76 (d, J=8.3 Hz, 1H), 7.64 (ddt, J=16.1, 6.4, 1.4 Hz, 2H), 7.58 (t, J=6.5 Hz, 1H), 6.66 (s, 2H).

### Synthesis of Compound 41d

41c (9.0 g, 17.81 mmol), an intermediate a1 (5.20 g, 14.85 mmol), ammonium acetate (45 g), and 90 mL acetic acid were added to a 250 ml single-necked flask and subjected to a reaction at 130° C. for 20 h under the protection of nitrogen. After the reaction was finished, the reaction liquid was extracted with EA/H₂O for several times to remove most of the acetic acid solution and the residual ammonium acetate solid, added with silica gel, subjected to a rotary evaporation, loaded into a column with pure HX, and eluted with HX/EA 40/1 for a first pass and with HX/THF 40/1 for a second pass. The remaining unpurified portion was further recrystallized with EA/CH₃OH for several times. 7.06 g of light yellow powder was obtained with a yield of 63%.

¹H NMR (400 MHz, CDCl₃) δ8.11 (dd, J=2.0, 1.4 Hz, 2H), 7.92 (dd, J=8.4, 1.6 Hz, 2H), 7.81 (dd, J=6.2, 1.5 Hz, 1H), 7.75 (d, J=8.4 Hz, 1H), 7.71-7.66 (m, 2H), 7.55 (td, J=7.4, 1.6 Hz, 1H), 7.52-7.46 (m, 2H), 7.43 (d, J=2.2 Hz, 2H), 7.38 (td, J=7.6, 1.1 Hz, 2H), 7.15 (ddd, J=8.6, 7.5, 1.1 Hz, 1H), 6.90 (dd, J=7.7, 1.2 Hz, 1H), 3.90 (s, 3H), 1.35 (s, 18H).

### Synthesis of Compound 41e

41d (6.5 g, 10.32 mmol), B₂Pin₂ (5.24 g, 20.64 mmol), Pd(dppf)Cl₂ (0.79 g, 1.03 mmol), KOAc (3.0 g, 30.96 mmol), and DMF (120 mL) were added to a 250 ml single-necked flask and subjected to a reaction at 85° C. for 16 h under the protection of nitrogen. After the reaction was finished, a rotary evaporation was performed to remove most of DMF. The crude product was extracted with EA and water, dried, subjected to a rotary evaporation, and subjected to a separation by column chromatography with EA/HX in 1/10 to 1/4 as an eluent. The impure portion was further recrystallized with EA/CH₃OH. 5.1 g of white powder was obtained with a yield of 73%.

¹H NMR (400 MHz, CDCl₃) δ 8.42 (d, J=1.9 Hz, 1H), 8.11-8.05 (m, 2H), 7.92 (dd, J=8.4, 1.6 Hz, 2H), 7.81 (dd, J=6.2, 1.5 Hz, 1H), 7.76-7.69 (m, 2H), 7.55 (td, J=7.4, 1.6 Hz, 1H), 7.49 (s, 2H), 7.43 (d, J=2.2 Hz, 2H), 7.38 (td, J=7.6, 1.1 Hz, 1H), 7.15 (ddd, J=8.6, 7.5, 1.1 Hz, 1H), 6.90 (dd, J=7.7, 1.2 Hz, 1H), 3.90 (s, 3H), 1.35 (s, 18H), 1.24 (s, 12H).

### Synthesis of Compound 41f

41e (5.0 g, 7.3 mmol), a2 (1.85 g, 6.64 mmol), Pd(PPh₃)₄ (0.382 g, 0.33 mmol), Cs₂CO₃ (6.4 g, 19.92 mmol), and toluene/ethanol/water (100/100/25 mL) were added to a 500 ml single-necked and subjected to a reaction at 90° C. for 48 h under the protection of nitrogen. After the reaction was finished, a rotary evaporation was performed to remove most of ethanol. The crude product was extracted with EA and water, dried, subjected to a rotary evaporation, subjected to a separation by column chromatography with EA/HX in 1/5 as an eluent, and then recrystallized with EA/CH₃OH. 3.8 g of white powder was obtained with a yield of 67%.

¹H NMR (400 MHz, CDCl₃) δ9.38 (d, J=1.7 Hz, 1H), 8.80 (d, J=4.4 Hz, 1H), 8.26 (d, J=2.2 Hz, 1H), 8.17-8.11 (m, 3H), 7.95-7.85 (m, 4H), 7.85-7.79 (m, 2H), 7.70 (dd, J=7.5, 1.4 Hz, 1H), 7.55 (td, J=7.4, 1.6 Hz, 1H), 7.52-7.44 (m, 3H), 7.43 (d, J=2.2 Hz, 2H), 7.41-7.31 (m, 3H), 7.27-7.20 (m, 1H), 7.15 (ddd, J=8.6, 7.5, 1.1 Hz, 1H), 6.90 (dd, J=7.7, 1.2 Hz, 1H), 3.90 (s, 3H), 1.35 (s, 18H).

### Synthesis of Compound 41g

41f (3.5 g, 4.4 mmol), iodobenzene (2.69 g, 13.2 mmol), Cu (144.1 mg, 2.2 mmol), CuI (418.9 mg, 2.2 mmol), o-phenanthroline (792.9 mg, 4.4 mmol), Cs₂CO₃ (4.30 g, 13.2 mmol), and xylene (100 mL) were added to a 250 ml single-necked flask and subjected to a reaction at 160° C. for 24 h under the protection of nitrogen. A small amount of iodobenzene and the catalyst was supplemented and then the reaction was continued for 48 h. After the reaction was finished, the reaction liquid was extracted with EA and water, dried, subjected to a rotary evaporation, subjected to a separation by column chromatography with EA/HX=1/4 as an eluent, and then pulped with CH₃OH at 80° C. for 3 h. 2.75 g of a white solid was obtained with a yield of 72%.

¹H NMR (400 MHz, CDCl₃) δ9.38 (d, J=1.7 Hz, 1H), 8.76 (d, J=4.5 Hz, 1H), 8.28 (d, J=2.2 Hz, 1H), 8.17-8.11 (m, 2H), 8.09 (dt, J=6.9, 0.7 Hz, 1H), 7.95-7.85 (m, 4H), 7.81 (dd, J=6.2, 1.5 Hz, 1H), 7.68 (ddd, J=14.5, 6.8, 1.4 Hz, 2H), 7.62 (dd, J=7.0, 0.7 Hz, 1H), 7.59-7.47 (m, 6H), 7.45-7.26 (m, 9H), 7.15 (ddd, J=8.6, 7.5, 1.1 Hz, 1H), 6.90 (dd, J=7.7, 1.2 Hz, 1H), 3.90 (s, 3H), 1.35 (s, 18H).

### Synthesis of Complex 41h

41g (3 g, 3.45 mmol), pyridine hydrochloride (15 g), and o-dichlorobenzene (3 mL) were added to a 250 ml single-necked flask and subjected to a reaction at 200° C. for 8 h under the protection of nitrogen. After the reaction was finished, the reaction liquid was extracted with DCM and water, dried, subjected to a rotary evaporation, subjected to separation by column chromatography with HX/DCM/EA=10/5/1 as an eluent, and then pulped with HX at 80° C. for 3 h. 1.77 g of a light yellow solid was obtained with a yield of 60%.

¹H NMR (400 MHz, CDCl₃) δ9.38 (d, J=1.7 Hz, 1H), 8.76 (d, J=4.5 Hz, 1H), 8.28 (d, J=2.2 Hz, 1H), 8.14 (dd, J=7.4, 1.8 Hz, 2H), 8.12-8.06 (m, 1H), 8.00 (dd, J=8.7, 1.2 Hz, 1H), 7.94-7.85 (m, 3H), 7.81 (dd, J=6.2, 1.5 Hz, 1H), 7.68 (ddd, J=14.5, 6.8, 1.4 Hz, 2H), 7.62 (dd, J=7.0, 0.7 Hz, 1H), 7.58-7.47 (m, 6H), 7.46-7.28 (m, 8H), 7.25 (td, J=8.0, 1.3 Hz, 1H), 7.02-6.94 (m, 2H), 1.35 (s, 18H).

### Synthesis of Complex 41

41h (1.5 g, 1.75 mmol), K₂PtCl₄ (0.79 g, 2.1 mmol), TBAB (112.8 mg, 0.35 mmol), and acetic acid (120 mL) were added to a 250 ml single-necked flask and subjected to a reaction at 130° C. for 48 h under the protection of nitrogen. During the reaction, the reaction liquid was gradually cloudy and a yellow solid was precipitated. After the reaction was finished, the reaction liquid was added with water to precipitate a solid and suction filtered. The filtered solid was extracted with DCM/water, subjected to a rotary evaporation, loaded into a column, eluted with DCM for a first pass to obtain a product which was subjected to a rotary evaporation and eluted with HX/DCM/EA=2/1/0.3 for a second pass. The obtained crude product was recrystallized with DCM/HX. 1.50 g of an orange-red solid was obtained with a yield of 81.79%.

¹H NMR (400 MHz, CDCl₃) δ 9.10 (d, J=8.9 Hz, 1H), 8.28 (d, J=2.1 Hz, 1H), 8.18-8.12 (m, 1H), 8.09 (dt, J=6.6, 0.8 Hz, 1H), 8.04 (d, J=8.4 Hz, 1H), 7.94 (dd, J=8.3, 1.2 Hz, 1H), 7.92-7.86 (m, 2H), 7.75 (dd, J=9.4, 2.3 Hz, 2H), 7.70 (d, J=2.2 Hz, 1H), 7.67 (dd, J=6.2, 1.7 Hz, 1H), 7.62 (dd, J=7.5, 1.5 Hz, 1H), 7.60-7.47 (m, 7H), 7.45-7.27 (m, 8H), 7.18 (dd, J=7.5, 1.3 Hz, 1H), 7.09 (ddd, J=8.4, 7.5, 1.3 Hz, 1H), 6.90 (d, J=2.9 Hz, 2H), 1.35 (s, 18H).

¹³C NMR (101 MHz, CDCl₃) δ 166.24, 152.13, 151.60, 148.10, 144.64, 144.12, 140.90, 140.24, 139.59, 139.32, 138.93, 138.92, 138.22, 137.16, 136.19, 134.35, 133.21, 132.84, 131.64, 131.57, 131.49, 130.53, 130.05, 128.67, 128.33, 128.06, 127.52, 127.50, 126.91, 126.87, 125.77, 125.15, 124.75, 123.93, 123.50, 123.47, 123.44, 122.86, 122.83, 122.63, 122.39, 121.69, 121.59, 120.81, 120.47, 117.66, 113.76, 112.54, 109.43, 34.96, 31.29.

ESI-MS (m/z): 1048.3 (M+1)

### Example 4

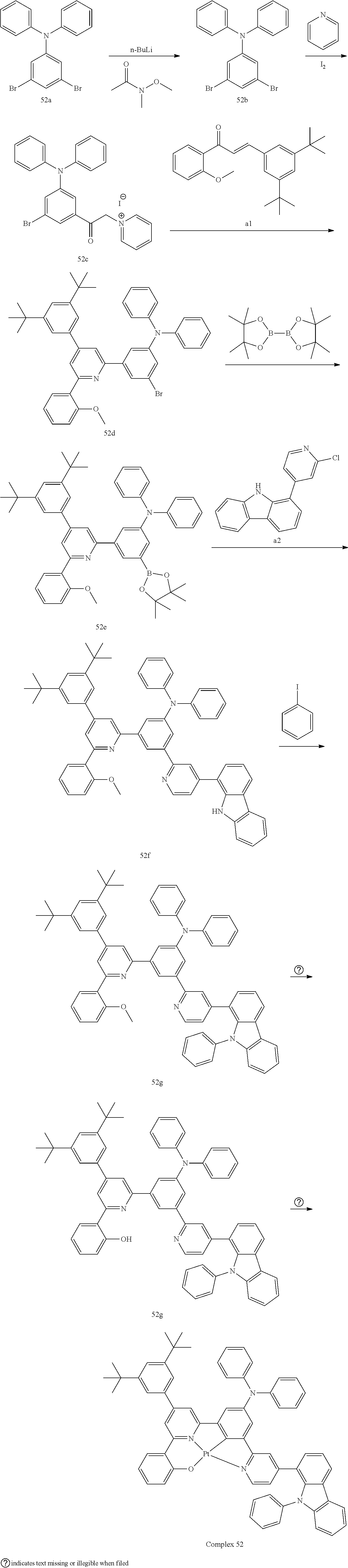

### Synthesis of Compound 52b

To a 250 ml single-neck flask, 52a (10.0 g, 24.8 mmol) and 100 mL diethyl ether were added at room temperature and N₂ was introduced. The reaction flask was placed in a low temperature reactor at −78° C. and kept for 30 min. After the temperature was stable, the reaction flask was slowly added dropwise with n-BuLi (17.4 mL, 27.28 mmol) through a dropping funnel and kept at −78° C. for 1 hour after the dropwise addition. Subsequently, the reaction flask was slowly added dropwise with N-methoxy-N-methylacetamide (2.81 g, 27.28 mmol) through the dropping funnel, kept for 30 min after the dropwise addition, warmed to room temperature, and subjected to stirring overnight at room temperature. After the reaction was finished, the reaction liquid was extracted with EA/H₂O for three times and subjected to a separation by silica gel column chromatography (with Hex/EA in 20/1 as an eluant).7.2 g of a yellow oil was obtained with a yield of 79.7%.

¹H NMR (400 MHz, CDCl₃) δ 7.84 (t, J=2.1 Hz, 1H), 7.59 (t, J=2.2 Hz, 1H), 7.44 (t, J=2.2 Hz, 1H), 7.28 (tt, J=7.7, 1.5 Hz, 4H), 7.14-7.08 (m, 4H), 7.04 (tt, J=7.6, 1.4 Hz, 2H), 2.61 (s, 3H).

### Synthesis of Compound 52c

52b (7.0 g, 19.1 mmol), elementary iodine (5.3 g, 21.0 mmol), and pyridine (60.4 g, 0.76 mol) were added to a 250 ml single-necked flask and subjected to a reaction at 130° C. for 16 h under the protection of nitrogen. After the reaction was finished, the reaction liquid was added with 200 ml ethyl acetate to produce a yellow precipitate and suction filtered. The crude product was pulped with 250 mL ethyl acetate at 80° C. for three times, suction filtered, and oven dried. 10.5 g of pink powder was obtained with a yield of 97%.

¹H NMR (400 MHz, DMSO) δ9.13 (dd, J=6.6, 1.3 Hz, 2H), 8.65 (tt, J=7.9, 1.3 Hz, 1H), 8.26-8.20 (m, 2H), 7.93 (t, J=2.2 Hz, 1H), 7.67 (t, J=2.2 Hz, 1H), 7.44 (t, J=2.2 Hz, 1H), 7.28 (tt, J=7.6, 1.5 Hz, 4H), 7.14-7.08 (m, 4H), 7.04 (tt, J=7.7, 1.4 Hz, 2H), 6.70 (s, 2H).

### Synthesis of Compound 52d

52c (10.0 g, 17.5 mmol), an intermediate a1 (5.12 g, 14.6 mmol), ammonium acetate (50 g), and 90 mL acetic acid were added to a 250 ml single-necked flask and subjected to a reaction at 130° C. for 20 h under the protection of nitrogen. After the reaction was finished, the reaction liquid was extracted with EA/H₂O for several times to remove most of the acetic acid solution and the residual ammonium acetate solids, added with silica gel, subjected to a rotary evaporation, loaded into a column with pure HX, and eluted with HX/EA in 40/1 for a first pass and with HX/THF in 40/1 for a second pass. The remaining unpurified portion was further recrystallized with EA/CH₃OH for several times. 8.03 g of light yellow powder was obtained with a yield of 66%.

¹H NMR (400 MHz, CDCl₃) δ8.09 (d, J=2.2 Hz, 1H), 7.99 (t, J=2.1 Hz, 1H), 7.95-7.89 (m, 2H), 7.50 (td, J=2.2, 0.7 Hz, 1H), 7.44-7.35 (m, 4H), 7.28 (tt, J=7.6, 1.5 Hz, 4H), 7.18-7.08 (m, 5H), 7.04 (tt, J=7.7, 1.4 Hz, 2H), 6.90 (dd, J=7.7, 1.2 Hz, 1H), 3.90 (s, 3H), 1.35 (s, 18H).

### Synthesis of Compound 52e

52d (7 g, 10.06 mmol), B₂Pin₂ (5.11 g, 20.12 mmol), Pd(dppf)Cl₂ (0.73 g, 1.0 mmol), KOAc (2.98 g, 30.36 mmol), and DMF (120 mL) were added to a 250 ml single-necked flask and subjected to a reaction at 85° C. for 16 h under the protection of nitrogen. After the reaction was finished, a rotary evaporation was performed to remove most of DMF. The crude product was extracted with EA and water, dried, subjected to a rotary evaporation, and subjected to a separation by column chromatography with EA/HX in 1/10 to 1/4 as an eluent. The impure portion was further recrystallized with EA/CH₃OH. 5.3 g of white powder was obtained with a yield of 71%.

¹H NMR (400 MHz, CDCl₃) δ 8.08 (d, J=2.2 Hz, 1H), 7.94-7.89 (m, 2H), 7.81 (t, J=2.2 Hz, 1H), 7.52-7.47 (m, 2H), 7.44-7.35 (m, 3H), 7.33-7.25 (m, 5H), 7.18-7.08 (m, 5H), 7.04 (tt, J=7.7, 1.4 Hz, 2H), 6.90 (dd, J=7.7, 1.2 Hz, 1H), 3.90 (s, 3H), 1.35 (s, 18H), 1.24 (s, 12H).

### Synthesis of Compound 52f

52e (5.0 g, 6.7 mmol), a2 (1.7 g, 6.1 mmol), Pd(PPh₃)₄ (0.382 g, 0.33 mmol), Cs₂CO₃ (6.1 g, 18.76 mmol), and toluene/ethanol/water (100/100/25 mL) were added to a 500 ml single-necked flask and subjected to a reaction at 90° C. for 48 h under the protection of nitrogen. After the reaction was finished, a rotary evaporation was performed to remove most of ethanol. The crude product was extracted with EA and water, dried, subjected to a rotary evaporation, subjected to a separation by column chromatography with EA/HX in 1/5 as an eluent, and then recrystallized with EA/CH₃OH. 4.2 g of white powder was obtained with a of 73%.

¹H NMR (400 MHz, CDCl₃) δ8.79 (d, J=4.5 Hz, 1H), 8.24 (d, J=2.2 Hz, 1H), 8.21 (t, J=2.2 Hz, 1H), 8.14 (ddd, J=7.7, 4.1, 1.0 Hz, 2H), 8.10 (d, J=2.2 Hz, 1H), 7.97-7.90 (m, 2H), 7.82 (dd, J=7.5, 0.8 Hz, 1H), 7.56 (dt, J=4.7, 2.2 Hz, 2H), 7.51-7.45 (m, 3H), 7.42 (d, J=2.1 Hz, 2H), 7.40-7.32 (m, 3H), 7.28 (dd, J=7.8, 7.0 Hz, 4H), 7.26-7.20 (m, 1H), 7.15 (ddd, J=8.6, 7.5, 1.1 Hz, 1H), 7.12-7.08 (m, 4H), 7.04 (tt, J=7.7, 1.4 Hz, 2H), 6.90 (dd, J=7.7, 1.2 Hz, 1H), 3.90 (s, 3H), 1.35 (s, 18H).

### Synthesis of Compound 52g

52f (4 g, 4.3 mmol), iodobenzene (2.63 g, 12.9 mmol), Cu (136.6 mg, 2.15 mmol), CuI (409.5 mg, 2.15 mmol), o-phenanthroline (774.9 mg, 4.3 mmol), Cs₂CO₃ (4.20 g, 12.9 mmol), and xylene (100 mL) were added to a 250 ml single-necked flask and subjected to a reaction at 160° C. for 24 h under the protection of nitrogen. A small amount of iodobenzene and the catalyst was supplemented and then the reaction was continued for 48 h. After the reaction was finished, the reaction liquid was extracted with EA and water, dried, subjected to a rotary evaporation, subjected to a separation by column chromatography with EA/HX=1/4 as an eluent, and then pulped with CH₃OH at 80° C. for 3 h. 2.9 g of a white solid was obtained with a yield of 72%.

¹H NMR (400 MHz, CDCl₃) δ8.75 (d, J=4.4 Hz, 1H), 8.24 (d, J=2.2 Hz, 1H), 8.21 (t, J=2.2 Hz, 1H), 8.17-8.13 (m, 1H), 8.11-8.06 (m, 2H), 7.95-7.89 (m, 2H), 7.67 (dd, J=6.2, 1.7 Hz, 1H), 7.62 (dd, J=7.0, 0.7 Hz, 1H), 7.56 (dt, J=4.7, 2.2 Hz, 2H), 7.54-7.47 (m, 4H), 7.45-7.41 (m, 4H), 7.41-7.26 (m, 9H), 7.15 (ddd, J=8.6, 7.5, 1.1 Hz, 1H), 7.11 (dd, J=7.1, 1.3 Hz, 4H), 7.04 (tt, J=7.7, 1.4 Hz, 2H), 6.90 (dd, J=7.7, 1.2 Hz, 1H), 3.90 (s, 3H), 1.35 (s, 18H).

### Synthesis of complex 52h

52g (2.5 g, 2.67 mmol), pyridine hydrochloride (12.5 g), and o-dichlorobenzene (2.5 mL) were added to a 250 ml single-necked flask and subjected to a reaction at 200° C. for 8 h under the protection of nitrogen. After the reaction was finished, the reaction liquid was extracted with DCM and water, dried, subjected to a rotary evaporation, subjected to a separation by column chromatography with HX/DCM/EA=10/5/1 as an eluent, and then pulped with HX at 80° C. for 3 h. 2 g of a light yellow solid was obtained with a yield of 81.3%.

¹H NMR (400 MHz, CDCl₃) δ 8.75 (d, J=4.4 Hz, 1H), 8.24 (d, J=2.2 Hz, 1H), 8.21 (t, J=2.2 Hz, 1H), 8.18-8.13 (m, 1H), 8.11-8.06 (m, 2H), 7.99 (dd, J=8.8, 1.3 Hz, 1H), 7.85 (d, J=2.2 Hz, 1H), 7.67 (dd, J=6.2, 1.7 Hz, 1H), 7.62 (dd, J=7.0, 0.7 Hz, 1H), 7.56 (dt, J=4.7, 2.2 Hz, 2H), 7.54-7.45 (m, 4H), 7.45-7.18 (m, 13H), 7.14-7.08 (m, 4H), 7.08-6.94 (m, 4H), 1.35 (s, 18H).

### Synthesis of complex 52

52h (1.5 g, 1.62 mmol), K₂PtCl₄ (0.71 g, 1.9 mmol), TBAB (104.4 mg, 0.324 mmol), and acetic acid (120 mL) was added to a 250 ml single-necked flask and subjected to a reaction at 130° C. for 48 h under the protection of nitrogen. During the reaction, the reaction liquid was gradually cloudy and a yellow solid was precipitated. After the reaction was finished, the reaction liquid was added with water to precipitate a solid and suction filtered. The filtered solid was extracted with DCM/water, subjected to a rotary evaporation, loaded into a column, and eluted with pure DCM for a first pass to obtain a product which was subjected to a rotary evaporation and eluted with HX/DCM/EA=2/1/0.3 for a second pass. The obtained crude product was further recrystallized with DCM/HX. 1.50 g of an orange-red solid was obtained with a yield of 83.1%.

¹H NMR (400 MHz, CDCl₃) δ 9.05 (d, J=8.9 Hz, 1H), 8.23 (d, J=2.2 Hz, 1H), 8.16-8.12 (m, 1H), 8.11-8.06 (m, 2H), 7.94 (dd, J=8.2, 1.2 Hz, 1H), 7.75 (dd, J=9.3, 2.3 Hz, 2H), 7.70-7.64 (m, 3H), 7.56-7.47 (m, 5H), 7.45-7.25 (m, 12H), 7.20-7.14 (m, 2H), 7.13-7.07 (m, 5H), 7.04 (tt, J=7.7, 1.4 Hz, 2H), 6.91 (s, 1H), 1.35 (s, 18H).

¹³C NMR (101 MHz, CDCl₃) δ 166.22, 152.05, 151.60, 147.81, 147.38, 146.01, 144.39, 142.70, 140.34, 139.59, 139.32, 138.96, 138.92, 137.14, 134.45, 134.12, 131.74, 131.64, 130.52, 130.07, 129.32, 128.33, 128.06, 126.91, 126.87, 125.95, 125.20, 125.16, 125.15, 124.75, 124.31, 123.93, 123.52, 123.50, 123.47, 123.44, 123.29, 122.86, 122.63, 122.10, 121.90, 121.69, 121.59, 120.81, 120.47, 113.76, 112.54, 34.96, 31.29.

ESI-MS (m/z): 1114.4 (M+1)

### Example 5

Under nitrogen atmosphere, fully dried samples of platinum complexes 23, 26, 41, and 52 were each weighted at approximately 5.0 mg and subjected to a heating scan at a rate of 10° C./min within a range of 25-800° C. The determined thermal decomposition temperatures (corresponding to a thermal weight loss of 0.5%) were 418.5° C., 472.6° C., 478.9° C., and 491° C., respectively, suggesting the excellent thermal stability of these complexes.

### Example 6

An organic light-emitting diode was prepared using the complex luminescent material of the present application, and the structure of the device is as shown in the FIGURE.

Firstly, a transparent conductive ITO glass substrate 10 (provided with an anode 20 thereon) was washed sequentially with a detergent solution and deionized water, ethanol, acetone, and deionized water, and then treated with oxygen plasma for 30 seconds.

Then, HATCN was evaporated onto ITO as a hole injection layer 30 with a thickness of 10 nm.

Then, the compound HT was evaporated to form a hole transport layer 40 with a thickness of 40 nm.

Then, a light-emitting layer 50 with a thickness of 20 nm was evaporated onto the hole transport layer. The light-emitting layer was formed of the platinum complex 23 (20%) in combination with CBP (80%).

Then, AlQ₃ was evaporated onto the light-emitting layer as an electron transport layer 60 with a thickness of 40 nm.

Finally, 1 nm LiF was evaporated as an electron injection layer 70 and 100 nm A1 was evaporated as a cathode 80 of the device.

### Example 7

An organic light-emitting diode was prepared by the method as described in Example 6, with the complex 23 replaced by the complex 26.

### Example 8

An organic light-emitting diode was prepared by the method as described in Example 6, with the complex 23 replaced by the complex 41.

### Example 9

An organic light-emitting diode was prepared by the method as described in Example 6, with the complex 23 replaced by the complex 52.

### Comparative Example 1

An organic light-emitting diode was prepared by the method as described in Example 6, with the complex 23 replaced by the complex Ref-1(CN110872325A).

### Comparative Example 2

An organic light-emitting diode was prepared by the method as described in Example 6, with the complex 23 replaced by the complex Ref-2 (Chem. Sci., 2014,5,4819).

### Comparative Example 3

An organic light-emitting diode was prepared by the method as described in Example 6, with the complex 23 replaced by complex Ref-3(CN110872325A).

### Comparative Example 4

An organic light-emitting diode was prepared by the method as described in Example 6, with the complex 23 replaced by complex Ref-4(CN110872325A).

HATCN, HT, AlQ₃, Ref-1, Ref-2, Ref-3, Ref-4, and CBP in the device have the following structural formulas:

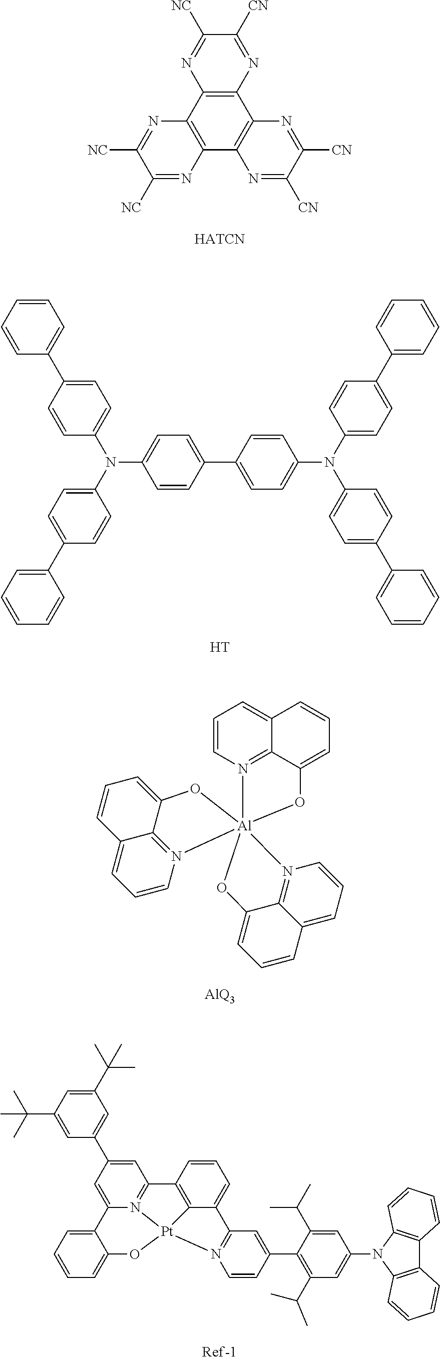

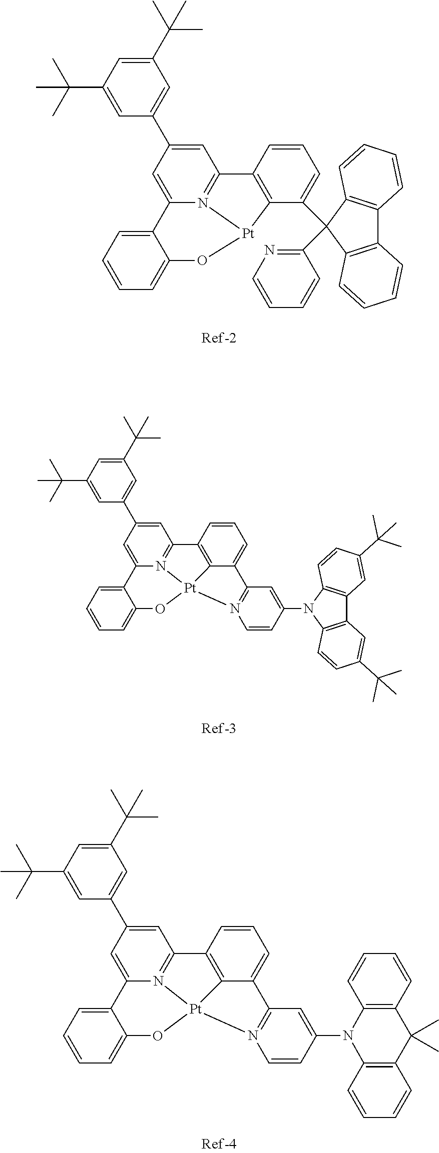

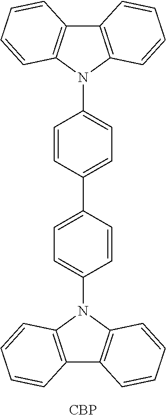

The device performances of the organic electroluminescent devices in Examples 3, Comparative example 1, Comparative example 2, Comparative example 3, and Comparative example 4 at a current density of 20 mA/cm² are listed in Table 1:

TABLE 1

| | | | | Device |
|---|---|---|---|---|
| | | Driving | Luminous | lifespan |
| Device No. | Complex | voltage | efficiency | (LT95) |
| Example 6 | Complex 23 | 1 | 1 | 1 |
| Example 7 | complex26 | 0.9 | 1.1 | 0.82 |
| Example 8 | complex41 | 1 | 1.2 | 0.71 |
| Example 9 | complex52 | 1.03 | 1.4 | 0.63 |
| Comparative | Ref-1 | 1.1 | 0.95 | 0.35 |
| Example 1 | | | | |
| Comparative | Ref-2 | 1.1 | 0.91 | 0.20 |
| Example 2 | | | | |
| Comparative | Ref-3 | 1.07 | 0.72 | 0.36 |
| Example 3 | | | | |
| Comparative | Ref-4 | 1.05 | 0.83 | 0.07 |
| Example 4 | | | | |

Note:
The properties of the device were tested with Example 6 as a benchmark, with individual indicators of properties of the device in Example 6 as 1. LT95 refers to a duration until the device's brightness diminishes to 95% of the initial brightness (10000 cd/m²).

From the data shown in Table 1, it can be seen that under the same condition, the platinum complex materials of the present application can be used to prepare the organic light-emitting diodes with lower driving voltages and higher luminous efficiencies. In addition, the device lifespans of the organic light-emitting diode based on the complexes of the present application are significantly longer as compared to the complex materials in the Comparative Examples, meeting the requirements on the luminescent materials in the display industry and having good industrialization prospects.

The various embodiments as described above are only used as examples, and are not intended to limit the scope of the present application. Other materials and structures may be used to replace the various materials and structures in the present application without departing from the spirit of the present application. It should be understood that various modifications and changes may be made by those skilled in the art according to the concept of the present application without creative effort. Therefore, all technical solutions obtained by those skilled in the art through analysis, inference, or partial research on basis of the existing technologies shall fall within the scope of protection defined by the claims.

What is claimed is:

1. A platinum complex, being a compound having a structure of Formula (I): 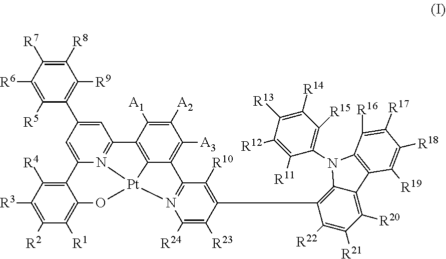
R¹ to R²⁴ are each independently selected from hydrogen, deuterium, halogen, amino, carboxyl, thioalkyl, cyano, sulfonyl, phosphino, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, a substituted or unsubstituted alkoxy having 1 to 20 carbon atoms, a substituted or unsubstituted aryl having 6 to 30 carbon atoms, or a substituted or unsubstituted heteroaryl having 3 to 30 carbon atoms, or any two adjacent substituents are linked or fused to form a ring;
A₁ to A₃ are each independently selected from hydrogen, deuterium, halogen, amino, carboxyl, thioalkyl, cyano, sulfonyl, phosphino, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, a substituted or unsubstituted alkoxy having 1 to 20 carbon atoms, an aryl having 6 to 30 carbon atoms, or a substituted or unsubstituted heteroaryl having 3 to 30 carbon atoms, or any two adjacent substituents are linked or fused to form a ring;
a heteroatom in the heteroaryl group includes one or more of N, S, or O; and
the “substituted” refers to substitution with halogen, deuterium, amino, cyano, or C1-C4 alkyl.

2. The platinum complex according to claim 1, wherein R¹ to R²⁴ are each independently selected from hydrogen, deuterium, halogen, amino, thioalkyl, cyano, a substituted or unsubstituted alkyl having 1 to 6 carbon atoms, a substituted or unsubstituted cycloalkyl having 3 to 6 ring carbon atoms, a substituted or unsubstituted alkenyl having 2 to 6 carbon atoms, a substituted or unsubstituted alkoxy having 1 to 6 carbon atoms, a substituted or unsubstituted aryl having 6 to 12 carbon atoms, or a substituted or unsubstituted heteroaryl having 3 to 6 carbon atoms;
A₁ to A₃ are each independently selected from hydrogen, deuterium, halogen, cyano, a substituted or unsubstituted alkyl having 1 to 20 carbon atoms, a substituted or unsubstituted cycloalkyl having 3 to 20 ring carbon atoms, a substituted or unsubstituted alkenyl having 2 to 20 carbon atoms, an aryl having 6 to 30 carbon atoms, or a substituted or unsubstituted heteroaryl having 3 to 6 carbon atoms.

3. The platinum complex according to claim 2, wherein R¹ to R²⁴ are each independently selected from hydrogen, deuterium, halogen, or a substituted or unsubstituted alkyl having 1 to 6 carbon atoms;
one or more of A₁ to A₃ are selected from halogen, cyano, a substituted or unsubstituted alkyl having 1 to 6 carbon atoms, a substituted or unsubstituted cycloalkyl having 3 to 12 ring carbon atoms, a substituted or unsubstituted alkenyl having 2 to 12 carbon atoms, an aryl having 6 to 30 carbon atoms, or a substituted or unsubstituted heteroaryl having 3 to 30 carbon atoms, and the rest are hydrogen.

4. The platinum complex according to claim 2, wherein R¹ to R²⁴ are each independently selected from hydrogen, deuterium, methyl, or tert-butyl;
one or more of A₁ to A₃ are selected from methyl, fluorine, cyano, tert-butyl, phenyl, cyano phenyl, or pyridyl, and the rest are hydrogen.

5. The platinum complex according to claim 1, wherein R¹ to R⁴ and R¹⁰ to R²⁴ of R¹ to R²⁴ are hydrogen.

6. The platinum complex according to claim 5, wherein at least one of R⁵ to R⁹ are not hydrogen.

7. The platinum complex according to claim 6, wherein R⁶ and R⁸ of R⁵ to R⁹ are not hydrogen, and R⁵ and R⁹ of R⁵ to R⁹ are hydrogen.

8. The platinum complex according to claim 1, being one of the following compounds:

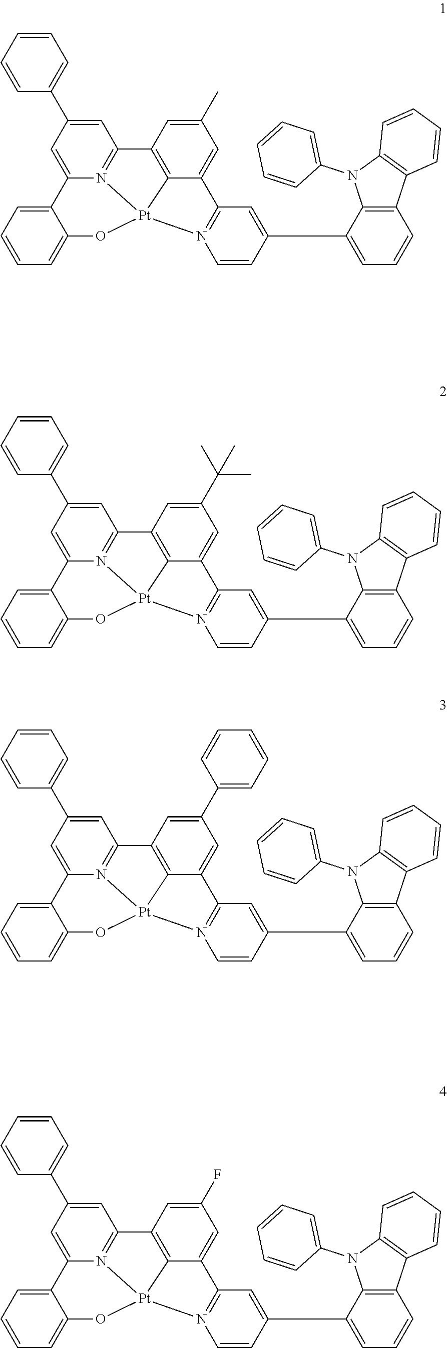

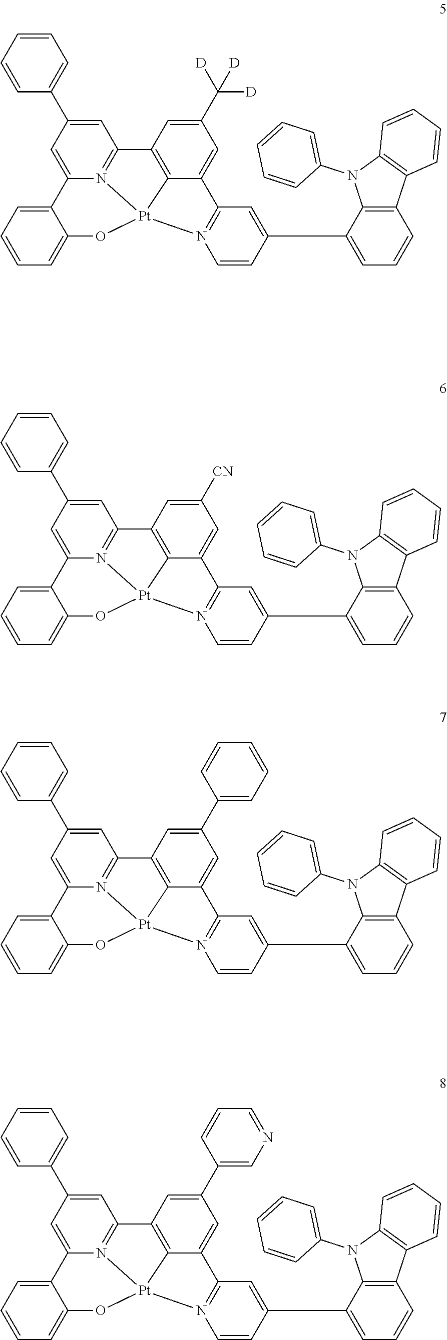

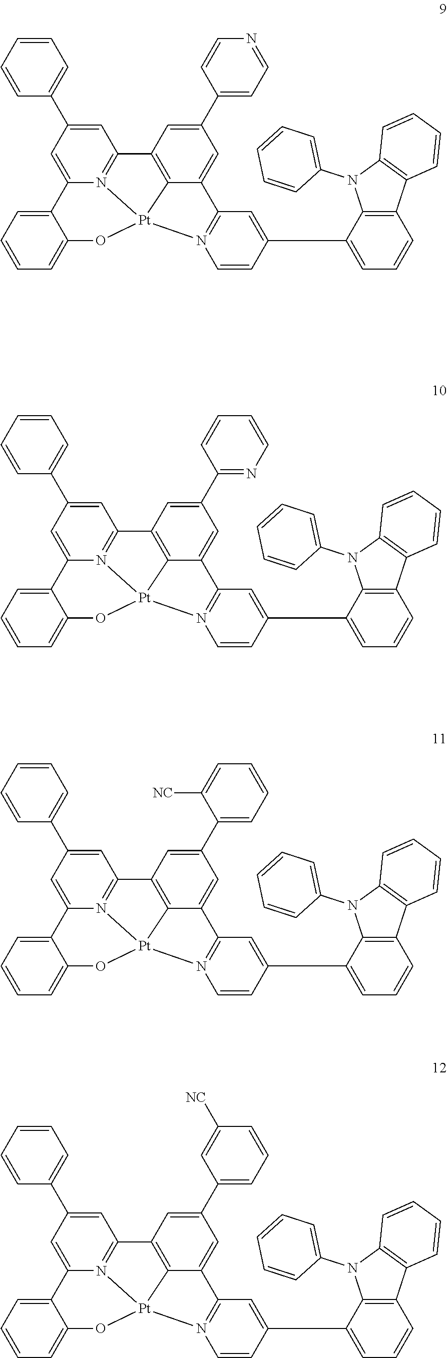

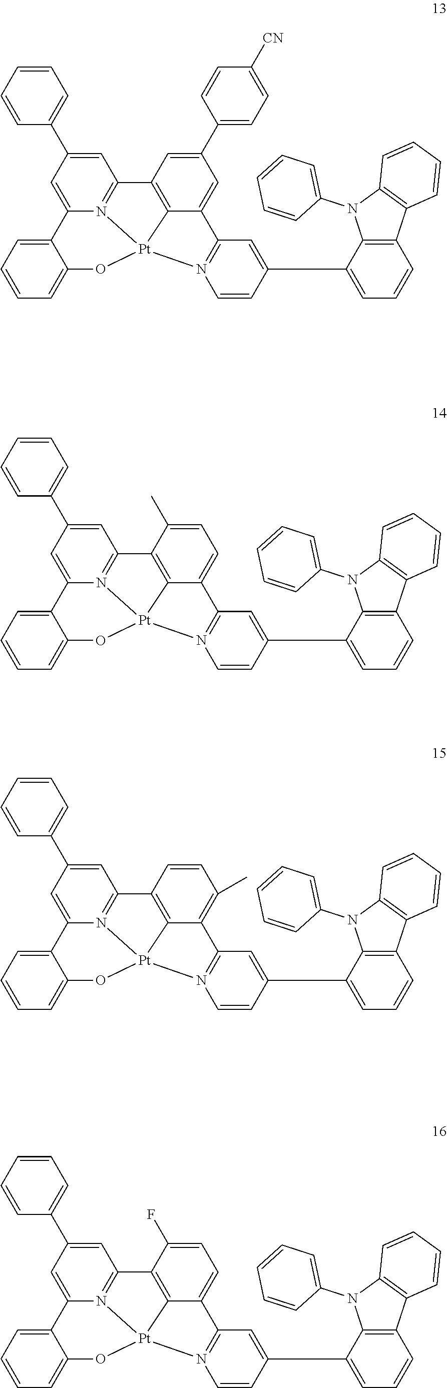

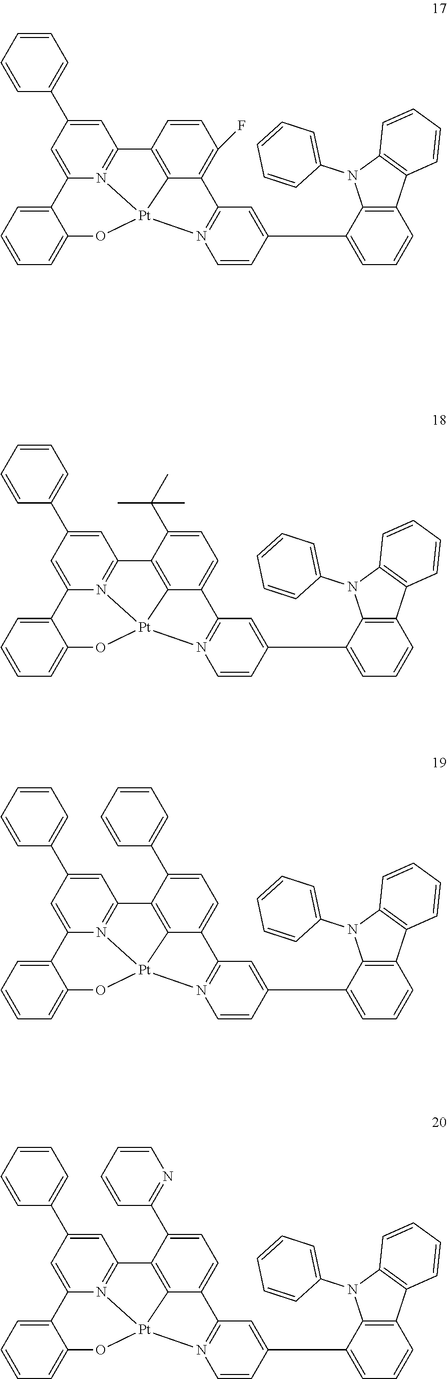

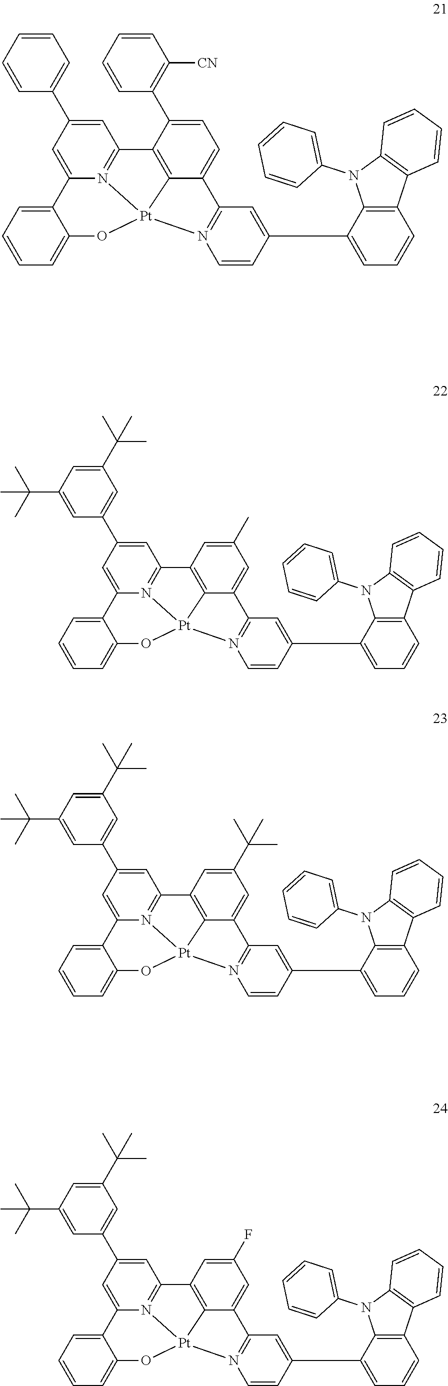

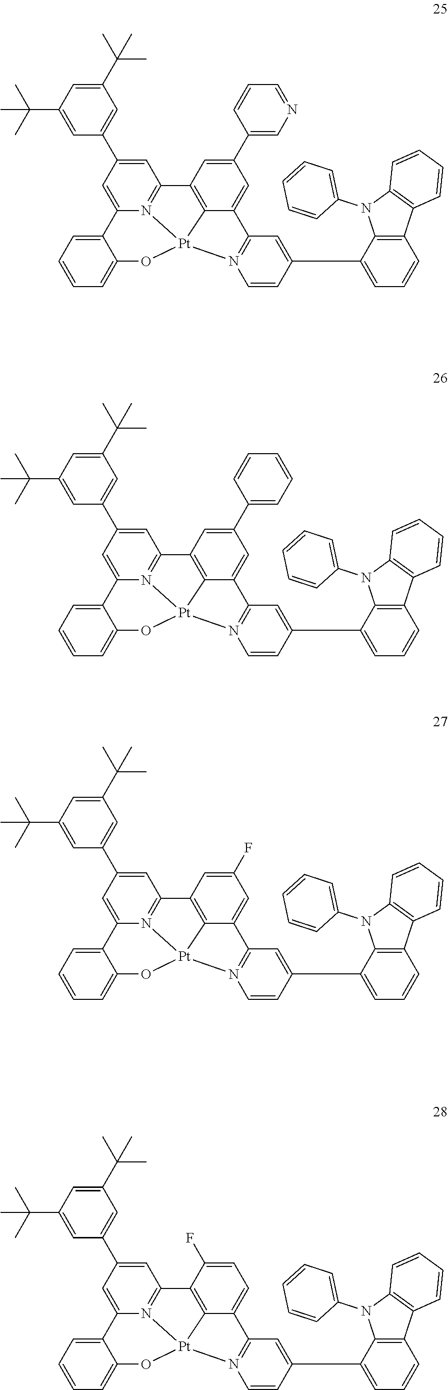

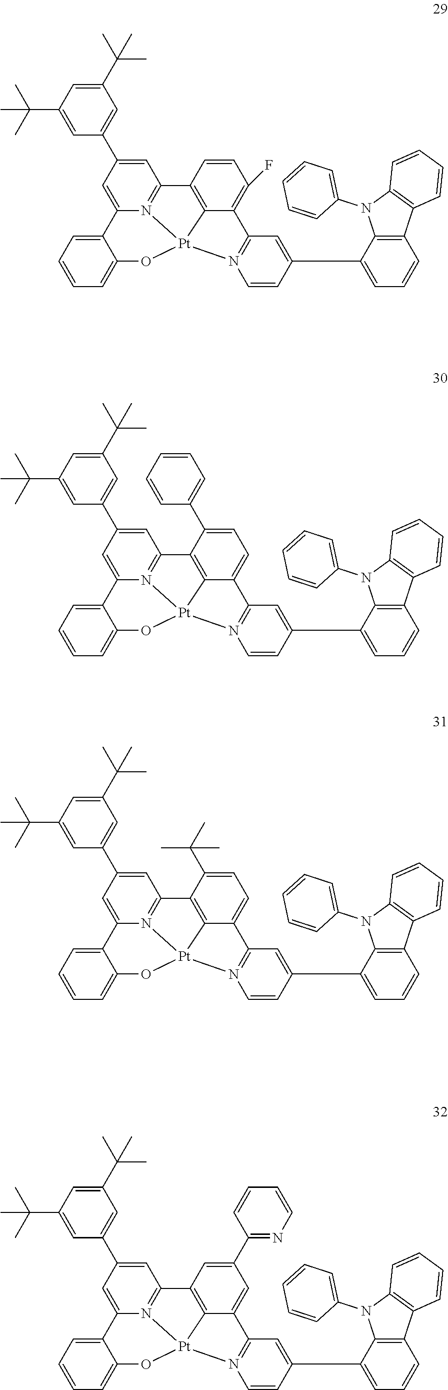

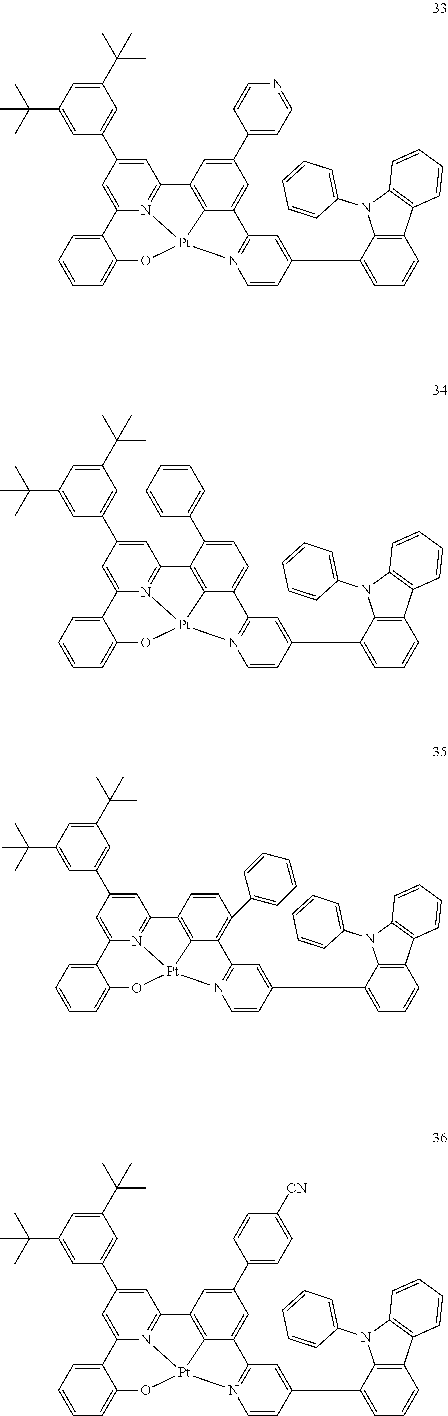

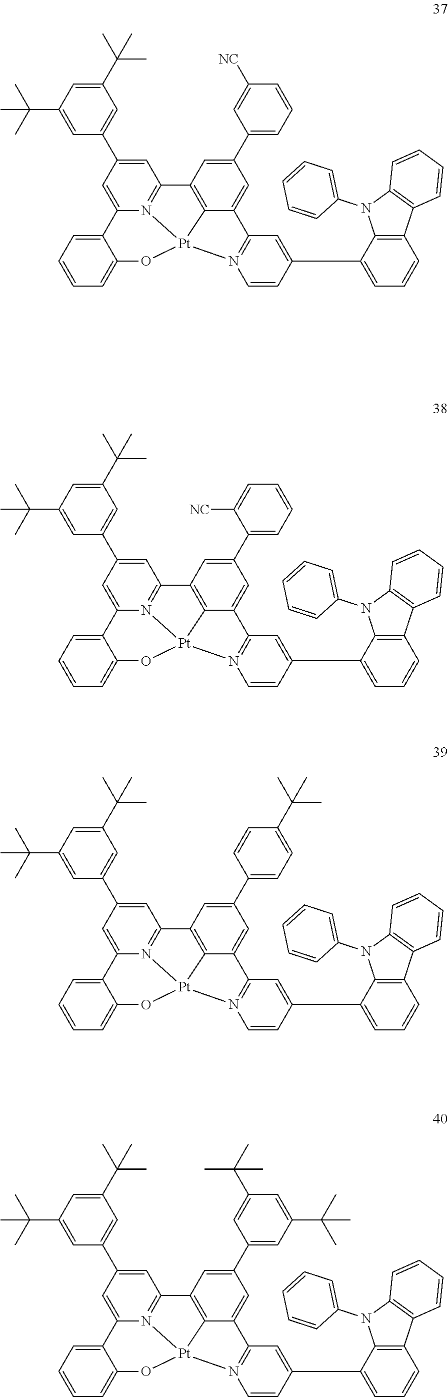

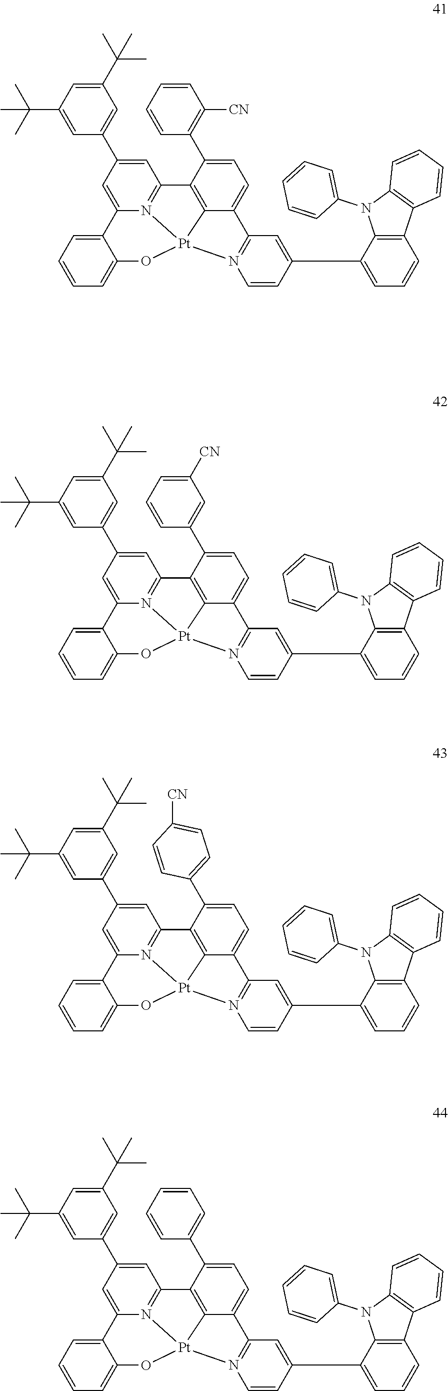

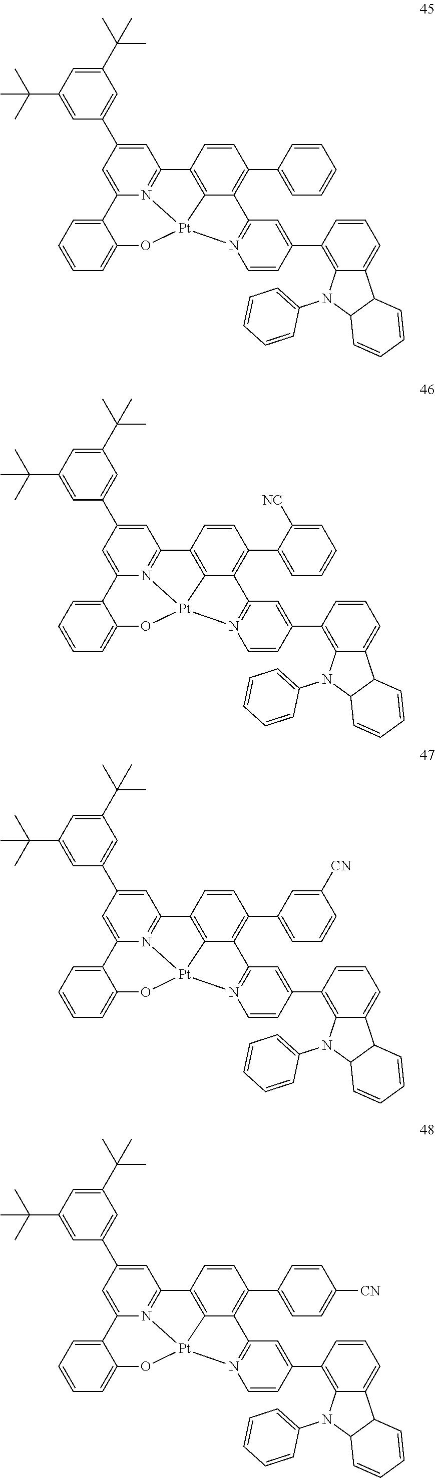

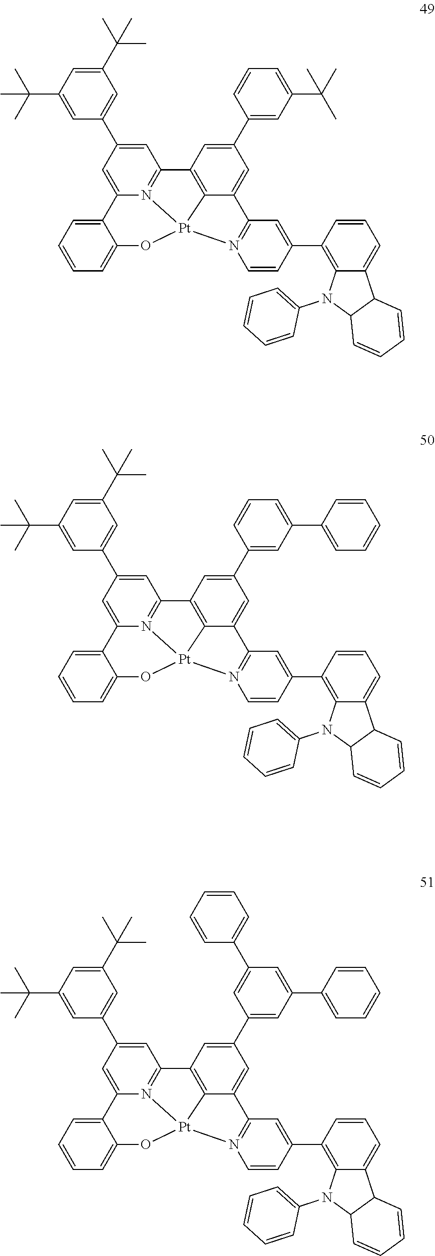

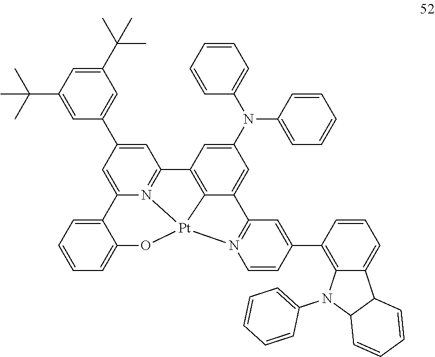

9. A precursor of the platinum complex according to claim 1, having the following structural formula:

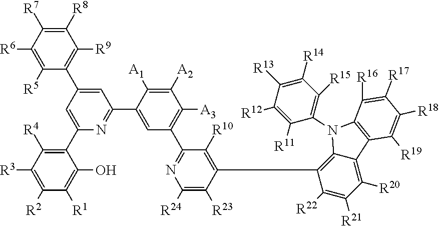

10. A device comprising the platinum complex according to claim 1, wherein the device is an organic light-emitting diode, an organic thin film transistor, an organic photovoltaic device, a luminescent electrochemical cell, or a chemical sensor.

11. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 1.

12. The organic light-emitting device according to claim 11, wherein the light-emitting layer comprises the platinum complex.

13. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 2.

14. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 3.

15. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 4.

16. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 5.

17. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 6.

18. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 7.

19. An organic light-emitting device, comprising a cathode, an anode, and an organic layer, wherein the organic layer includes one or more of a hole injection layer, a hole transport layer, a light-emitting layer, a hole blocking layer, an electron injection layer, or an electron transport layer; and the organic layer comprising the platinum complex according to claim 8.
